(19) United States
(12) Patent Application Publication
(10) Pub. No.: US 2022/0267467 A1
(43) Pub. Date: Aug. 25, 2022

(54) POLYPEPTIDE COMPLEX FOR CONJUGATION AND USE THEREOF

(71) Applicant: WUXI BIOLOGICS IRELAND LIMITED, Co Louth (IE)

(72) Inventors: Mingzhi JIN, Shanghai (CN); Xiaoyue CHEN, Shanghai (CN); Yue ZHANG, Shanghai (CN); Chen ZHANG, Shanghai (CN); Li YIN, Shanghai (CN); Jiexing CAI, Shanghai (CN); Jun WANG, Shanghai (CN); Weichang ZHOU, Shanghai (CN)

(21) Appl. No.: 17/628,008

(22) PCT Filed: Jul. 17, 2020
(86) PCT No.: PCT/CN2020/102585

§ 371 Date: Jan. 18, 2022

(30) Foreign Application Priority Data
Jul. 19, 2019 (CN) PCT/CN2019/096849

(51) Int. Cl. C07K 16/32; C07K 16/28; A61P 35/00; A61K 47/68

(52) U.S. Cl. CPC C07K 16/32; C07K 16/2863; A61P 35/00; A61K 47/6803; C07K 16/2887; C07K 2317/53; C07K 2317/55

(57) ABSTRACT

The disclosure generally relates to the fields of immunology, cell biology, molecular biology and medicine. More particularly, it concerns polypeptide complex for conjugation and use thereof. A polypeptide complex comprising, from N-terminus to C-terminus, a Fab domain operably linked to a hinge region is provided, wherein the Fab domain and the hinge region are derived from different IgG isotypes, or part thereof. The polypeptide complex further comprises a Fc polypeptide which is operably linked to the hinge region. The present disclosure provides an antibody drug conjugate comprising a polypeptide complex of the present disclosure. A pharmaceutical composition comprising an antibody drug conjugate of the present disclosure and a pharmaceutically acceptable carrier or excipient, a method of preparing the antibody drug conjugate, the use of the polypeptide complex in the manufacture of the antibody drug conjugate, a method of treating a condition in a subject in need thereof with a therapeutically effective amount of the antibody drug conjugate are also provided. The subject inventions according to the present disclosure provide improved payload-antibody ratio of antibody bio-conjugation, in particular for therapeutic applications.

### FIELD OF THE INVENTION

The present invention relates generally to the fields of immunology, cell biology, molecular biology and medicine. More particularly, it concerns polypeptide complex for conjugation and use thereof.

### BACKGROUND

Antibodies are multifunctional immunoglobulins carrying unique binding specificity for a target antigen and a series of antigen-independent immunological interactions, which allow them to play critical roles in the immune system. Many currently used biological therapeutics, diagnosis and research agent are antibodies directed against antigens that are involved in the pathological, immunological or biological mechanism of interest.

In recent years, extensive efforts have been made to develop antibody conjugates which have payload attached. In case of an antibody-drug conjugate (ADC), ADC is comprised by an antibody for targeting, a linker for drug attachment and a high potent payload as an effector. Antibody or its relevant forms brings the cytotoxic drugs to antigen-expressing cells or other target cells by antibody-antigen interaction. At the same time, the toxicity of drugs after conjugated to the antibody is dramatically reduced. Thus, ADC enlarges the therapeutic window by reducing Minimum Effect Dose (MED) and elevating Maximum Tolerance Dose (MTD). Mylotarg, Adcetris, Kadcyla, Besponsa, Polivy, Padcev, Enhertu and Trodelvy are examples of ADC drugs approved by FDA.

A successful ADC development depends on antibody selection, linker-payload selection, manner of linker-payload conjugation and conjugation process development. Cysteine thiols in antibody as strong nucleophiles are ideal reaction groups for conjugation. Since cysteine residues exist as disulfide bonds in native form of antibodies, reduction of disulfide bonds between light-heavy chain and heavy-heavy chain in antibody provides perfect free cysteine thiols for conjugation. Numerous conjugation methods have been developed in the art to address the opportunities and challenges afforded by having the preferred payload-antibody ratio (PAR) and conjugation positions. Ideally, moderate number of payloads should be attached to one antibody, resulting in heterogeneous ADC product. Conjugated product with low PAR has insufficient therapeutic efficacy and product with high PAR shows high toxicity as well as instability. As a result, the heterogeneity of ADC does harm in enlarging therapeutic window. Thus, efforts such as antibody engineering were made to improve the homogeneity of ADC product.

One approach is using point mutation on antibody to introduce amino acid with high reactivity residue for conjugation. Thiomab™ technology was developed by Genentech with inducing Cysteine mutation on antibody (Jagath R Junutula, et al., Site-specific conjugation of a cytotoxic drug to an antibody improves the therapeutic index, Nature Biotechnology, 2008, 26 (8): 925-932). Conjugation with Thiomab happened at the engineered Cysteine residue after reduction, resulting in high homogeneous conjugate product. Non-natural amino acid (NNAA) technology was also used for homogeneous conjugate production. For instance, keto group or azido moiety were induced to antibody by unnatural amino acid as conjugation site (Jun Y. Axup, et al., Synthesis of site-specific antibody-drug conjugates using unnatural amino acids, PNAS, 2012, 109(40): 16101-16106; Michael P. VanBrunt, et al., Genetically Encoded Azide Containing Amino Acid in Mammalian Cells Enables Site-Specific Antibody-Drug Conjugates Using Click Cycloaddition Chemistry, Bioconjugate Chem., 2015, 26(11): 2249-2260), also resulted in highly homogeneous product due to specific reaction.

Site mutation-based method has several disadvantages. First of all, mutation site needs to be carefully selected, otherwise both antibody's stability and conjugation efficiency will be affected. Secondly, expression level of the antibody with site mutation is usually very low, which may be a problem in the Chemistry, Manufacturing and Controls (CMC) stage.

Another approach is introducing short polypeptide tags as conjugation site which can be recognized by enzymes. A glutamine tag (LLQG) as mTG recognition motif (Pavel Strop, et al., Location Matters: Site of Conjugation Modulates Stability and Pharmacokinetics of Antibody Drug Conjugates, Chemistry & Biology, 2013, 20(2): 161-167), LPETG as sortase A recognition motif (Roger R. Beerli, et al., Sortase Enzyme-Mediated Generation of Site-Specifically Conjugated Antibody Drug Conjugates with High In Vitro and In Vivo Potency, PLOS ONE, 2015, 10(7): e0131177), and LCxPxR as formylglycine-generating enzyme (FGE) recognition motif (Peng Wu, et al., Site-specific chemical modification of recombinant proteins produced in mammalian cells by using the genetically encoded aldehyde tag, PNAS, 2009, 106(9): 3000-3005) are used for conjugation, resulting in highly homogeneous conjugate products with drugs attached at polypeptide tags.

The disadvantage of short polypeptide tags is similar to site mutation-based method. Insert site for polypeptide tags needs to be screened, and usually available sites are limited for polypeptide tags. And expression titer of tagged antibodies is one of the challenges in using this strategy, as well.

The most straightforward approach of producing an antibody conjugate is utilizing the thiol group of natural cysteines in the antibody heavy and light chain polypeptide. Thiol group, as a strong nucleophilic reagent, provides fast and efficient conjugation reaction in aqueous phase. In the FDA approved ADC drugs, Adcetris and Polivy, Monomethyl auristatin E (MMAE) is conjugated at cysteine residues generated from partial reduction of interchain disulfide bonds, via the reaction between thiol groups on cysteine residues and maleimide group in the linker for MMAE. Here partial but not full reduction is preferred, since hydrophobicity of drugs and hindrance when all cysteine residues are attached causes the instability of ADC drugs in plasma. However, homogeneity of the product after partial reduction is very poor. As reported, an average of four free thiols after partial reduction for IgG1 type antibodies is preferred as ADCs with drug-antibody ratio (DAR) as 4 exhibit the best therapeutic index in vivo.

There are many similarities and differences with regard to the disulfide bond structures among the IgG subclasses, IgG1, IgG2, IgG3, and IgG4. Taking IgG1 and IgG4 which are most commonly used as therapeutic biologics for example, IgG1 and IgG4 both have their two heavy chains connected by two disulfide bonds and contains a total of 12 intra-chain disulfide bonds; however the light chain of IgG1 is linked to the heavy chain by a disulfide bond between the last residue of the light chain and the fifth cysteine residue of the heavy chain, while the light chain of IgG4 is linked to the heavy chain by a disulfide bond between the last cysteine residue of the light chain and the third cysteine residue of the heavy chain (See FIG. 1). In general, the level of solvent exposure is different between intra-chain and inter-chain disulfide bonds. Intra-chain disulfide bonds are all buried between the secondary structures of each domain and are not solvent exposed. The inter-chain disulfide bond, including the inter-heavy-heavy-chain disulfide bond for IgG1 and IgG4, and the inter-heavy-light-chain disulfide bond for IgG1, which is located in the hinge region are highly solvent exposed. The inter-heavy-light-chain disulfide bond for IgG4, which is located between the less accessible interface of VH and CH1 domains is not as much solvent exposed as a result. The solvent exposure difference between different disulfide bonds has important implications for antibody bio-conjugation because the exposed cysteine residues are considered more reactive than non-exposed ones (Hongcheng Liu & Kimberly May, Disulfide bond structures of IgG molecules: Structural variations, chemical modifications and possible impacts to stability and biological function, Mabs, 2012, 4(1): 17-23). There are experiment evidences that both IgG1 inter-heavy-light-chain and inter-heavy-heavy-chain disulfide bond are highly reactive.

Hinge region is a flexible linker between the Fab and the Fc of an antibody. Length and flexibility of the hinge region vary extensively among the IgG subclasses, IgG1, IgG2, IgG3, and IgG4. Taking IgG1 and IgG4 which are most commonly used as therapeutic biologics for example, the hinge region of IgG1 comprises 15 amino acids and is very flexible, while IgG4 has a shorter hinge with only 12 amino acids (Gestur Vidarsson, et al., IgG subclasses and allotypes: from structure to effector functions, Front. Immunol., 2014, 5: 520). Wild-type IgG1 and IgG4 differ by one amino acid in the core hinge region (226-229 by EU numbering): Cys-Pro-Pro-Cys in IgG1 and Cys-Pro-Ser-Cys in IgG4. Natural IgG4 presents an equilibrium between inter- and intra-chain cysteine disulfide bonds at the core hinge region, resulting in observable heavy chain arm exchange and the presence of IgG4 half molecules post secretion. S228P mutation for IgG4 has been confirmed to markedly stabilize the covalent interaction between IgG4 heavy-chains by preventing natural arm exchange (S. Angal, et al., A single amino acid substitution abolishes the heterogeneity of chimeric mouse/human (IgG4) antibody, Molecular Immunology, 1993, 30(1): 105-108; John-Paul Silva, et al., The S228P Mutation Prevents in Vivo and in Vitro IgG4 Fab-arm Exchange as Demonstrated using a Combination of Novel Quantitative Immunoassays and Physiological Matrix Preparation, Journal of Biological Chemistry, 2015, 290(9): 5462-5469), thus widely applied in IgG4 antibody development and production. The S228P mutation results in a poly-proline helix in the IgG4 hinge (5 Pro in the lower hinge), which when combined with the shorter IgG4 hinge length, will further restrict its flexibility compared to the IgG1 hinge (3 Pro in the lower hinge). The flexibility difference between different hinges has important implications for antibody bio-conjugation because the cysteine residues located in a flexible hinge fragment are considered more reactive than the ones located in a rigid hinge. There are experiment evidences that both S228P IgG4 inter-heavy-light-chain and inter-heavy-heavy-chain disulfide bonds are weakly reactive.

The disadvantage of utilizing natural cysteines for antibody conjugation is that the similarities of reactivity between four inter-chain disulfide bonds in IgG1 and IgG4, results in highly heterogeneous conjugation products. And as described, this heterogeneity narrows therapeutic window of conjugate drug in clinical use. For instance, ADC produced by partial reduction of native interchain disulfide bonds in IgG1 antibodies results in a mixture of products with normal distribution. Species with best therapeutic index, with conjugation number as 4 (PAR4), only occupies 40% in total mixture. Species with low conjugate number (PAR0 and PAR2) lack therapeutic efficacy while species with high conjugate number (PAR6 and PARE) exhibit high toxicity and instability (Kevin J. Hamblett, et al., Effects of Drug Loading on the Antitumor Activity of a Monoclonal Antibody Drug Conjugate, Clinical Cancer Research, 2004, 10(20): 7063-7070; Yilma T. Adem, et al., Auristatin Antibody Drug Conjugate Physical Instability and the Role of Drug Payload, Bioconjugate Chem., 2014, 25(4): 656-664). Heterogeneity of product with partial reduction of IgG4 antibody is even higher, and lots of antibody remain un-reduced when the level of full reduced antibody already gets high (FIG. 1).

Thus, there remains a need for improving the PAR of antibody bio-conjugation, in particular for therapeutic applications, that minimizes some or all the disadvantages mentioned above.

### SUMMARY OF THE INVENTION

The present disclosure provides polypeptide complex for conjugation and use thereof.

In a first aspect, the present disclosure provides a polypeptide complex comprising, from N-terminus to C-terminus, a Fab domain operably linked to a hinge region, wherein the Fab domain and the hinge region or part thereof are derived from different IgG isotypes, or part thereof.

In certain embodiments, the polypeptide complex is or comprises an immunoglobulin protein. In certain embodiments, the polypeptide complex is or comprises an antibody.

In certain embodiments, the hinge region, or part thereof, is a human IgG1, IgG2, IgG3 or IgG4 hinge region, or part thereof.

In certain embodiments, the hinge region, or part thereof, is a human IgG1 or IgG4 hinge region, or part thereof.

In certain embodiments, the hinge region, or part thereof, is a human IgG1 hinge, or part thereof. In certain embodiments, the hinge region or part thereof is of IgG1 isotype, while the Fab domain is of IgG4 isotype.

In certain embodiments, the hinge region or part thereof comprises (a) the sequence as set forth in DKTHTCPPCP (SEQ ID NO: 1) or a fragment thereof, or (b) a sequence having at least 85% of identity to (a), or (c) a variant of (a) or (b), wherein the variant has a mutation or mutations being selected from the group consisting of insertion, deletion and substitution, or the variant comprises a non-naturally occurring amino acid residue or non-naturally occurring amino acid residues.

In certain embodiments, the hinge region, or part thereof, comprises (a) the sequence as set forth in EPKSDKTHTCPPCP (SEQ ID NO: 2) or EPKDKTHTCPPCP (SEQ ID NO: 3), or (b) a sequence having at least 85% of identity to (a), or (c) a variant of (a) or (b), wherein the variant has a mutation or mutations being selected from the group consisting of insertion, deletion and substitution, or the variant comprises a non-naturally occurring amino acid residue or non-naturally occurring amino acid residues.

In certain embodiments, the hinge region or part thereof comprises (a) the sequence as set forth in any one of SEQ ID NOs: 12 to 14 or a fragment thereof, or (b) a sequence having at least 85% of identity to (a), or (c) a variant of (a) or (b), wherein the variant has a mutation or mutations being selected from the group consisting of insertion, deletion and substitution, or the variant comprises a non-naturally occurring amino acid residue or non-naturally occurring amino acid residues.

In certain embodiments, the hinge region, or part thereof, is a human IgG4 hinge, or part thereof. In certain embodiments, the hinge region or part thereof is of IgG4 isotype, while the Fab domain is of IgG1 isotype.

In certain embodiments, the hinge region, or part thereof, comprises (a) the sequence as set forth in EPKSCESKYGPPCPPCP (SEQ ID NO: 4) or a fragment thereof, (b) a sequence having at least 85% of identity to (a), or (c) a variant of (a) or (b), wherein the variant has a mutation or mutations being selected from the group consisting of insertion, deletion and substitution, or the variant comprises a non-naturally occurring amino acid residue or non-naturally occurring amino acid residues.

In certain embodiments, the hinge region, or part thereof, comprises (a) the sequence as set forth in EPKSCSKYGPPCPPCP (SEQ ID No. 5), or EPKSCKYGPPCPPCP (SEQ ID No. 6), or EPKSCYGPPCPPCP (SEQ ID No. 7), or EPKCESKYGPPCPPCP (SEQ ID No. 11) or (b) a sequence having at least 85% of identity to (a), or (c) a variant of (a) or (b), wherein the variant has a mutation or mutations being selected from the group consisting of insertion, deletion and substitution, or the variant comprises a non-naturally occurring amino acid residue or non-naturally occurring amino acid residues.

In certain embodiments, the hinge region, or part thereof, comprises (a) the sequence as set forth in EPKSCSKYGHTCPPCP (SEQ ID No. 8), or EPKSCSKYGHPCPPCP (SEQ ID No. 9), or EPKSCSKYGPTCPPCP (SEQ ID No. 10), or (b) a sequence having at least 85% of identity to (a), or (c) a variant of (a) or (b), wherein the variant has a mutation or mutations being selected from the group consisting of insertion, deletion and substitution, or the variant comprises a non-naturally occurring amino acid residue or non-naturally occurring amino acid residues.

In certain embodiments, the hinge region or part thereof comprises (a) the sequence as set forth in any one of SEQ ID NOs: 15-17 or a fragment thereof, (b) a sequence having at least 85% of identity to (a) or (c) a variant of (a) or (b), wherein the variant has a mutation or mutations being selected from the group consisting of insertion, deletion and substitution, or the variant comprises a non-naturally occurring amino acid residue or non-naturally occurring amino acid residues.

In another aspect, the present disclosure includes the polypeptide complex further comprising a Fc polypeptide which is operably linked to the hinge region or the polypeptide complex further comprising an additional polypeptide which is operably linked to the hinge region.

In certain embodiments, the Fc polypeptide is a human IgG1, IgG2, IgG3 or IgG4 Fc polypeptide.

In certain embodiments, Fc polypeptide is a human IgG1 or IgG4 Fc polypeptide.

In a further related aspect, the present disclosure includes an antibody drug conjugate comprising a polypeptide complex of the present disclosure.

In a related aspect, the present disclosure includes a pharmaceutical composition comprising an antibody drug conjugate of the present disclosure and a pharmaceutically acceptable carrier or excipient.

In a still further related aspect, the present disclosure includes a kit comprising a polypeptide complex of the present disclosure or an antibody drug conjugate of the present disclosure or a pharmaceutical composition of the present disclosure. Such a kit can be used in for research purposes or used as a therapeutic or diagnostic agent or a prophylactic therapeutic agent.

In another aspect, the present disclosure includes a method of preparing the antibody drug conjugate of the present disclosure, comprising:

providing the polypeptide complex of any one of the present disclosure;

reacting a maleimido or haloacetyl moiety with free thiol group in cysteine residue generated by reduction of interchain disulfide bonds via Michael addition reaction.

In certain embodiments, the free thiol group is generated by partial reduction of interchain disulfide bonds with mild reducing reagent such as TCEP or DTT, preferably the partial reduction is carried out in a buffer with pH range from about 4.0 to 8.0, with reducing agent (e.g., TCEP)/mAb ratio from about 3 to 10, reaction temperature from about 4° C. to 37° C., and for a reduction time from about 1 hr to 24 hr.

In certain embodiments, the free thiol group is generated by partial reduction of interchain disulfide bonds with mild reducing reagent such as TCEP or DTT. In certain embodiments, the partial reduction is carried out in a buffer with pH range from about 4.0 to 8.0 (e.g., pH 5.0 to 7.0, pH 5.0 to 6.0, pH 5.5 or pH 6.0), at a reducing agent/mAb ratio from about 1 to 20, 1 to 15, 1 to 10, 1 to 5, 3 to 20, 3 to 16, 3 to 6 or 4 to 8, a reaction temperature from about 4° C. to 37° C., 4° C. to 20° C., 4° C. to 15° C., 4° C. to 10° C. or 15° C. to 37° C., and/or for a reduction time from about 1 hr to 24 hr, 2 to 16 hr, 2 to 5 hr or 3 to 5 hr.

In certain embodiments, the partial reduction may be carried out at a temperature ranging from about 15° C. to 37° C., and/or at a reducing agent/mAb ratio ranging from about 3 to 6, wherein the polypeptide complex has a hinge or part thereof derived from an IgG1 hinge or an IgG4 hinge and optionally, an IgG1 or IgG4 Fc polypeptide. In certain embodiments, the polypeptide complex has a hinge comprising a sequence as set forth in any one of SEQ ID NOs: 1 to 3 and 12 to 14. In certain embodiments, the polypeptide complex has a hinge comprising a sequence as set forth in any one of SEQ ID NOs: 15 to 17.

In certain embodiments, the partial reduction is carried out at a temperature ranging from about 4° C. to 25° C., preferably about 4° C. to 20° C., 4° C. to 15° C. or 4 to 10° C. and/or at a reducing agent/mAb ratio ranging from about 1 to 20, preferably about 1 to 15, 3 to 16, 3 to 8, 1 to 6 or 3 to 5, wherein the polypeptide complex has a hinge or part thereof derived from a hinge having the structure of formula II (vide infra) and optionally, an IgG1 or IgG4 Fc polypeptide. In certain embodiments, the polypeptide complex has a hinge comprising a sequence as set forth in any one of SEQ ID NOs: 4 to 11.

In certain embodiments, the conjugation is carried out in a buffer with pH range from about 4.0 to 8.0, organic additive (e.g., organic solvent or organic co-solvent) from about 0.0% to 20.0% by weight, drug/mAb ratio from about 7 to 20, reaction temperature from about 4° C. to 37° C., and conjugation time from about 1 hr to 4 hr.

In another aspect, the present disclosure includes use of the polypeptide complex of the present disclosure for manufacturing an antibody drug conjugate.

In another aspect, the present disclosure includes a method of treating a condition in a subject in need thereof, comprising administrating to the subject a therapeutically effective amount of the antibody drug conjugate of the present disclosure.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from the detailed description.

### BRIEF DESCRIPTION OF THE FIGURES

The following drawings form part of the present specification and are included to further demonstrate certain aspects of the invention. The invention may be better understood by reference to one or more of these drawings in combination with the detailed description of specific embodiments presented herein.

FIG. 1 shows the structures of IgG1 and IgG4 and HIC-HPLC result of antibody-drug conjugate produced by IgG1 and IgG4 antibodies with partial reduction and conjugation via the reaction of free thiol groups and MC-vc-PAB-MMAE.

FIG. 2 shows the structure of antibody 886-5 and HIC-HPLC result after conjugated to MC-vc-PAB-MMAE. By the engineering of hinge region, homogeneity of ADC produced by this IgG4 based antibody showed impressive improvement.

FIG. 3 shows the structure of antibody 886-8 and HIC-HPLC result after conjugated to MC-vc-PAB-MMAE. By the engineering of hinge region and combination of IgG1-Fab and IgG4-Fc, homogeneity of ADC produced by this antibody showed impressive improvement.

FIG. 4 shows the structure of antibody 886-13 and HIC-HPLC result after conjugated to MC-vc-PAB-MMAE. By the engineering of hinge region, homogeneity of ADC produced by this IgG1 based antibody showed impressive improvement.

FIG. 5 shows the structure of antibody 886-29 and HIC-HPLC result after conjugated to MC-vc-PAB-MMAE. By the engineering of hinge region, homogeneity of ADC produced by this IgG4 based antibody showed impressive improvement.

FIG. 6 shows the structure of antibody 886-34 and HIC-HPLC result after conjugated to MC-vc-PAB-MMAE. By the engineering of hinge region, homogeneity of ADC produced by this antibody showed impressive improvement

FIG. 7 shows the structure of antibody 886-16 and HIC-HPLC, PLRP-HPLC result after conjugated to MC-vc-PAB-MMAE. Characterization of 886-16-MMAE shows 886-16-MMAE can be used for in vitro and in vivo study.

FIG. 8 shows the structure of antibody 886-19 and HIC-HPLC, PLRP-HPLC result after conjugated to MC-vc-PAB-MMAE. Characterization of 886-19-MMAE shows 886-19-MMAE can be used for in vitro and in vivo study.

FIG. 9 shows the structure of antibody 886-17 and HIC-HPLC, PLRP-HPLC result after conjugated to MC-vc-PAB-MMAE. Characterization of 886-17-MMAE shows 886-17-MMAE can be used for in vitro and in vivo study.

FIG. 10 shows the structure of antibody 886-20 and HIC-HPLC, PLRP-HPLC result after conjugated to MC-vc-PAB-MMAE. Characterization of 886-20-MMAE shows 886-20-MMAE can be used for in vitro and in vivo study.

FIG. 11 shows the structure of antibody 886-18 and HIC-HPLC, PLRP-HPLC result after conjugated to MC-vc-PAB-MMAE. Characterization of 886-18-MMAE shows 886-18-MMAE can be used for in vitro and in vivo study.

FIG. 12 shows the structure of antibody 886-21 and HIC-HPLC, PLRP-HPLC result after conjugated to MC-vc-PAB-MMAE. Characterization of 886-21-MMAE shows 886-21-MMAE can be used for in vitro and in vivo study.

FIG. 13 shows the Cytotoxicity of MMAE conjugated ADCs on HCC1954 cells, HCC827 cells and Raji cells. IC₅₀ value of ADCs shows MMAE conjugated ADCs have high potency in inhibiting cell growth.

FIG. 14 shows pharmaceutical kinetic profile of 886-16-MMAE and 886-19-MMAE compared to Trastuzumab-MMAE in rat. Clearance rate of total antibody and conjugated antibody (ADC) in plasma was shown by dashed line and solid line, respectively.

### Definitions

The articles “a,” “an,” and “the” are used herein to refer to one or to more than one (i.e., to at least one) of the grammatical object of the article. By way of example, “a polypeptide complex” means one polypeptide complex or more than one polypeptide complex.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

The word “exemplary” is used herein to mean “serving as an example, instance, or illustration.” Any aspect described herein as “exemplary” is not necessarily to be construed as preferred or advantageous over other aspects.

Throughout this disclosure, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this disclosure to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The terms “polypeptide,” “peptide,” and “protein” are used interchangeably herein to refer to a polymer of amino acid residues, or an assembly of multiple polymers of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an alpha-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. An alpha-carbon refers to the first carbon atom that attaches to a functional group, such as a carbonyl. A beta-carbon refers to the second carbon atom linked to the alpha-carbon, and the system continues naming the carbons in alphabetical order with Greek letters. Amino acid mimetics refers to chemical compounds that have a structure that is different from the general chemical structure of an amino acid, but that functions in a manner similar to a naturally occurring amino acid. The term “protein” typically refers to large polypeptides. The term “peptide” typically refers to short polypeptides. Polypeptide sequences are usually described as the left-hand end of a polypeptide sequence is the amino-terminus (N-terminus); the right-hand end of a polypeptide sequence is the carboxyl-terminus (C-terminus). “Polypeptide complex” as used herein refers to a complex comprising one or more polypeptides that are associated to perform certain functions. In certain embodiments, the polypeptides are immune-related.

The term “antibody” as used herein encompasses any immunoglobulin, monoclonal antibody, polyclonal antibody, multispecific antibody, or bispecific (bivalent) antibody that binds to a specific antigen. A native intact antibody comprises two heavy chains and two light chains. Each heavy chain consists of a variable region (“HCVR”) and a first, second, and third constant region (CH1, CH2 and CH3), while each light chain consists of a variable region (“LCVR”) and a constant region (CL). Mammalian heavy chains are classified as α, δ, ε, γ, and μ, and mammalian light chains are classified as λ, or κ. The antibody has a “Y” shape, with the stem of the Y consisting of the second and third constant regions of two heavy chains bound together via disulphide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light (L) chain CDRs including LCDR1, LCDR2, and LCDR3, heavy (H) chain CDRs including HCDR1, HCDR2, HCDR3). CDR boundaries for antibodies may be defined or identified by the conventions of Kabat, Chothia, or Al-Lazikani (Al-Lazikani, B., Chothia, C., Lesk, A. M., J. Mol. Biol., 273(4), 927 (1997); Chothia, C. et al., J Mol. Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., National Institutes of Health, Bethesda, Md. (1991)). The three CDRs are interposed between flanking stretches known as framework regions (FRs), which are more highly conserved than the CDRs and form a scaffold to support the hypervariable loops. Each HCVR and LCVR comprises four FRs, and the CDRs and FRs are arranged from amino terminus to carboxy terminus in the order: FR1, CDR1, FR2, CDR2, FR3, CDR3, FR4. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain). Accordingly, in context of the present invention, a particular IgG isotype, e.g, “IgG1” or “IgG1 isotype”, refers to IgG isotypes of the defined subclass, and different IgG isotypes refer to IgG isotypes of different subclasses.

The term “variable domain” with respect to an antibody as used herein refers to an antibody variable region or a fragment thereof comprising one or more CDRs. Although a variable domain may comprise an intact variable region (such as HCVR or LCVR), it is also possible to comprise less than an intact variable region yet still retain the capability of binding to an antigen or forming an antigen-binding site.

The term “antigen-binding moiety” as used herein refers to an antibody fragment formed from a portion of an antibody comprising one or more CDRs, or any other antibody fragment that binds to an antigen but does not comprise an intact native antibody structure. Examples of antigen-binding moiety include, without limitation, a variable domain, a variable region, a diabody, a Fab, a Fab′, a F(ab′)₂, an Fv fragment, a disulphide stabilized Fv fragment (dsFv), a (dsFv)₂, a bispecific dsFv (dsFv-dsFv′), a disulphide stabilized diabody (ds diabody), a multispecific antibody, a camelized single domain antibody, a nanobody, a domain antibody, and a bivalent domain antibody. An antigen-binding moiety is capable of binding to the same antigen to which the parent antibody binds. In certain embodiments, an antigen-binding moiety may comprise one or more CDRs from a particular human antibody grafted to a framework region from one or more different human antibodies. For more and detailed formats of antigen-binding moiety are described in Spiess et al, 2015 (Supra), and Brinkman et al., mAbs, 9(2), pp. 182-212 (2017), which are incorporated herein by their entirety.

“Fab” refers to the portion consisting of a single light chain (both variable and constant regions) associating to the variable region and first constant region of a single heavy chain by a disulphide bond in an immunoglobulin (e.g., an antibody). In certain embodiments, the constant regions of both the light chain and heavy chain are replaced with TCR constant regions. The Fab portion is responsible for various antigen binding.

“Fab”′ refers to a fragment that includes an antibody light chain covanlantly bound to a portion of an heavy chain, which consists the variable region (VH) and the first (CH1) constant region and a portion of the hinge region.

“Fc” refers to the fragment consisting of the second (CH2) and the third (CH3) optionally as well as a fourth (CH4, as in the case of IgM) constant regions of a first heavy chain bound to the second and the third optionally as well as a fourth constant regions of a second heavy chain in an immunoglobulin (e.g., an antibody), or refers to the fragment consisting of a portion of the hinge region, the second (CH2) and the third (CH3) optionally as well as a fourth (CH4, as in the case of IgM) constant regions of a first heavy chain bound to a portion of the hinge region, the second and the third optionally as well as a fourth constant regions of a second heavy chain in an immunoglobulin (e.g., an antibody). The Fc portion of the antibody is responsible for various effector functions such as ADCC, and CDC, but does not function in antigen binding.

“Hinge region” in terms of an antibody includes the portion of a heavy chain molecule that joins the CH1 domain to the CH2 domain. This hinge region comprises approximately 12-62 amino acid residues and is flexible, thus allowing the two N-terminus antigen binding regions to move independently.

“CH2 domain” as used herein refers to includes the portion of a heavy chain molecule that extends, e.g., from about amino acid 244 to amino acid 360 of an IgG antibody using conventional numbering schemes (amino acids 244 to 360, Kabat numbering system; and amino acids 231-340, EU numbering system; see Kabat, E., et al., U.S. Department of Health and Human Services, (1983)).

The “CH3 domain” extends from the CH2 domain to the C-terminus of the IgG molecule and comprises approximately 108 amino acids. Certain immunoglobulin classes, e.g., IgM, further include a CH4 region.

“Fv” with regard to an antibody refers to the smallest fragment of the antibody to bear the complete antigen binding site. An Fv fragment consists of the variable domain of a single light chain bound to the variable domain of a single heavy chain. A number of Fv designs have been provided, including dsFvs, in which the association between the two domains is enhanced by an introduced disulphide bond; and scFvs can be formed using a peptide linker to bind the two domains together as a single polypeptide. Fvs constructs containing a variable domain of a heavy or light immunoglobulin chain associated to the variable and constant domain of the corresponding immunoglobulin heavy or light chain have also been produced. Fvs have also been multimerised to form diabodies and triabodies (Maynard et al., Annu Rev Biomed Eng 2 339-376 (2000)).

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

An “antigen” or “Ag” as used herein refers to a compound, composition, peptide, polypeptide, protein or substance that can stimulate the production of antibodies or a T cell response in cell culture or in an animal, including compositions (such as one that includes a cancer-specific protein) that are added to a cell culture (such as a hybridoma), or injected or absorbed into an animal. An antigen reacts with the products of specific humoral or cellular immunity (such as an antibody), including those induced by heterologous antigens.

An “epitope” or “antigenic determinant” refers to the region of an antigen to which a binding agent (such as an antibody) binds. Epitopes can be formed both from contiguous amino acids (also called linear or sequential epitope) or noncontiguous amino acids juxtaposed by tertiary folding of a protein (also called configurational or conformational epitope). Epitopes formed from contiguous amino acids are typically arranged linearly along the primary amino acid residues on the protein and the small segments of the contiguous amino acids can be digested from an antigen binding with major histocompatibility complex (MHC) molecules or retained on exposure to denaturing solvents whereas epitopes formed by tertiary folding are typically lost on treatment with denaturing solvents. An epitope typically includes at least 3, and more usually, at least 5, about 7, or about 8-10 amino acids in a unique spatial conformation.

The term “specific binding” or “specifically binds” as used herein refers to a non-random binding reaction between two molecules, such as for example between an antibody and an antigen. In certain embodiments, the polypeptide complex and the bispecific polypeptide complex provided herein specifically bind an antigen with a binding affinity (K_{D}) of ≤10⁻⁶ M (e.g., ≤5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤2×10⁻⁹ M, ≤10⁻⁹ M, or ≤10⁻¹⁰ M). K_{D} as used herein refers to the ratio of the dissociation rate to the association rate (k_{off}/kₒₙ), may be determined using surface plasmon resonance methods for example using instrument such as Biacore.

The term “operably link” or “operably linked” refers to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

A hinge is a region of consecutive amino acid residues that connect the C-terminus of the CH1 to the N-terminus of the CH2 domain of an immunoglobulin. In human IgG1, the hinge region runs from residue 216 to 230 by EU numbering. In human IgG4, the hinge region runs from residue from 219 to 230 by EU numbering.

The term “substitution” with regard to amino acid residue as used herein refers to naturally occurring or induced replacement of one or more amino acids with another in a peptide, polypeptide or protein. Substitution in a polypeptide may result in diminishment, enhancement, or elimination of the polypeptide's function.

Substitution can also be “conservative substitution” with reference to amino acid sequence, which refers to replacing an amino acid residue with a different amino acid residue having a side chain with similar physiochemical properties or substitution of those amino acids that are not critical to the activity of the polypeptide. For example, conservative substitutions can be made among amino acid residues with nonpolar side chains (e.g., Met, Ala, Val, Leu, and Ile, Pro, Phe, Trp), among residues with uncharged polar side chains (e.g., Cys, Ser, Thr, Asn, Gly and Gln), among residues with acidic side chains (e.g., Asp, Glu), among amino acids with basic side chains (e.g., His, Lys, and Arg), among amino acids with beta-branched side chains (e.g., Thr, Val and Ile), among amino acids with sulfur-containing side chains (e.g., Cys and Met), or among residues with aromatic side chains (e.g., Trp, Tyr, His and Phe). In certain embodiments, substitutions, deletions or additions can also be considered as “conservative substitution.” The number of amino acids that are inserted or deleted can be in the range of about 1 to 5. Conservative substitution usually does not cause significant change in the protein conformational structure, and therefore could retain the biological activity of a protein.

The term “mutation” or “mutated” with regard to amino acid residue as used herein refers to substitution, insertion, deletion or addition of an amino acid residue.

As used herein, a “homologue sequence” and “homologous sequence” are used interchangeably and refer to polynucleotide sequences (or its complementary strand) or amino acid sequences that have sequences identity of at least 80% (e.g. at least 85%, 88%, 90%, 91%, 92%, 93%, 94%, 95%, 96%, 97%, 98%, 99%) to another sequences when optionally aligned.

The term “subject” or “individual” or “animal” or “patient” as used herein refers to human or non-human animal, including a mammal or a primate, in need of diagnosis, prognosis, amelioration, prevention and/or treatment of a disease or disorder. Mammalian subjects include humans, domestic animals, farm animals, and zoo, sports, or pet animals such as dogs, cats, guinea pigs, rabbits, rats, mice, horses, swine, cows, bears, and so on.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

The present disclosure provides polypeptide complexes comprising, from N-terminus to C-terminus, a Fab domain operably linked to a hinge region, wherein the Fab domain and the hinge region or part thereof are derived from different IgG isotypes, or part thereof. The inventors unexpectedly found that such difference results in an improved payload-antibody ratio (PAR) during bio-conjugation using such polypeptide complexes, because it leads to a differential accessibility of reducing agent to the inter-chain disulfide bond. Accordingly, the polypeptide complexes according to the present disclosure provides a significantly improved homogeneity of product, especially in terms of enrichment of products having a PAR of 4, when being used for preparing and/or being incorporated in an ADC. In another aspect, the polypeptide complexes according to the present disclosure are also found to have an advantageous pharmaceutical kenektic and/or pharmaceutical dynamic properties.

Polypeptide Complex

Provided herein are novel polypeptide complexes comprising, from N-terminus to C-terminus, a Fab domain operably linked to a hinge region, wherein the Fab domain and the hinge region or part thereof are derived from different IgG isotypes, or part thereof.

In one embodiment, the polypeptide complex or part thereof comprises at least two heavy chains and two light chains, which have their two heavy chains connected by two disulfide bonds located in the hinge region. The hinge region, or part thereof, is a human IgG1, IgG2, IgG3 or IgG4 hinge region, or part thereof.

In one embodiment, by swapping the hinge region of IgG1 and IgG4, or part thereof, immunoglobulin at their natural structural positions on the C-terminus of Fab, such difference results in an improved payload-antibody ratio (PAR) during bio-conjugation, because it leads to a differential accessibility of reducing agent to the inter-chain disulfide bond. Additional advantages of the polypeptide complexes and constructs provided herein will become more evident in the following disclosure below.

In context of the present invention, the Fab domain may derive from any antibody, especially those that are clinically relevant. In some embodiments, the Fab domain derives from an antibody that specifically binds to a tumor antigen (TA), such as a tumor specific antigen (TSA) and a tumor-associated antigen (TAA). Examples of the tumor antigen include, but are not limited to, CD20, CD38, CD123, ROR1, ROR2, BCMA, PSMA, SSTR2, SSTR5, CD19, FLT3, CD33, PSCA, ADAM 17, CEA, Her2, EGFR, EGFR-vIII, CD30, FOLR1, GD-2, CA-IX, Trop-2, CD70, CD38, mesothelin, EphA2, CD22, CD79b, GPNMB, CD56, CD138, CD52, CD74, CD30, CD123, RON, and ERBB2. Examples of TA-specific antibodies include, but are not limited to, Trastuzumab (like in Examples 9 and 10 described below) Rituximab (like in Examples 11 and 12), Cetuximab (like in Examples 13 and 14), Bevacizumab, Panitumumab, Alemtuzumab, Matuzuma, Gemtuzumab, Polatuzumab, Inotuzumab, etc.

Hinge Region

Hinge region is a flexible linker between the Fab and the Fc of antibody. Length and flexibility of the hinge region varies extensively among the IgG subclasses, IgG1, IgG2, IgG3, and IgG4. Taking IgG1 and IgG4 which are most commonly used as therapeutic biologics for example, the hinge region of IgG1 comprises 15 amino acids (e.g., EPKSCDKTHTCPPCP (SEQ ID NO: 18)) and is very flexible, while IgG4 has a shorter hinge with only 12 amino acids (Gestur Vidarsson, et al., IgG subclasses and allotypes: from structure to effector functions, Frontiers in Immunology, 20 Oct. 2014, 5:520). Wild-type IgG1 and IgG4 differ by one amino acid in the core hinge region (226-229 by EU numbering): Cys-Pro-Pro-Cys in IgG1 and Cys-Pro-Ser-Cys in IgG4. Natural IgG4 presents an equilibrium between inter- and intra-chain cysteine disulfide bonds at the core hinge region, resulting in observable heavy chain arm exchange and the presence of IgG4 half molecules post secretion. S228P mutation for IgG4 (e.g., ESKYGPPCPPCP (SEQ ID NO: 19)) has been confirmed to markedly stabilize the covalent interaction between IgG4 heavy-chains by preventing natural arm exchange, thus widely applied in IgG4 antibody development and production. The S228P mutation results in a poly-proline helix (PPCPPCP) in the IgG4 hinge, which when combined with the shorter IgG4 hinge length, will further restrict its flexibility compared to the IgG1 hinge. The flexibility difference between different hinges has important implications for antibody bio-conjugation because the cysteine residues located in a flexible hinge fragment are considered more reactive than the ones located in a rigid hinge. There are experiment evidence that both S228P IgG4 inter-heavy-light-chain and inter-heavy-heavy-chain disulfide bond are weakly reactive.

In some embodiments, a Fab domain is operably linked to a hinge region, wherein the hinge region or part thereof is derived from an IgG1, or part thereof, the Fab domain is derived from an IgG4. By swapping the hinge region of IgG1 and IgG4 immunoglobulin at their natural structural positions on the C-terminus of Fab, such difference results in an improved payload-antibody ratio (PAR) during bio-conjugation due to a differential accessibility of reducing agent to the inter-chain bond, optionally, the disulfide bond.

In some embodiments, the modified hinge region comprises a sequence having the following formula (I):

X₁ X₂ X₃ X₄ X₅ X₆ X₇ X₈ X₉ X₁₀ CPPCP (I)

Wherein, X₁=null or E; X₂=null or P; X₃=null or K; X₄=null or S or E; X₅=null or C or S, preferably null; X₆=D or K; X₇=K or Y; X₈=T or G; and/or X₉X₁₀=HT, HP, PT or PP, Preferably PT or PP.

In some embodiments, the modified hinge region comprises a sequence having the following formula (II):

EPKx₁C x₂ x₃ x₄ x₅ x₆ x₇ x₈ CPPCP (II)

Wherein, x₁=null or S; x₂=null or E or S, preferably null; x₃=null or S or C; x₄=null or K or D; x₅=Y or K; x₆=G or T; and/or x₇x₈=PP, PT, HP or HT.

In one or more embodiments, exemplary modified hinge sequences are provided in the below Table 1.

TABLE 1
Exemplary modified hinge sequences of the
present disclosure

| Hinge Sequence | SEQ ID NO. |
|---|---|
| DKTHTCPPCP | 1 |
| EPKSDKTHTCPPCP | 2 |
| EPKDKTHTCPPCP | 3 |
| EPKSCESKYGPPCPPCP | 4 |
| EPKSCSKYGPPCPPCP | 5 |
| EPKSCKYGPPCPPCP | 6 |
| EPKSCYGPPCPPCP | 7 |
| EPKSCSKYGHTCPPCP | 8 |
| EPKSCSKYGHPCPPCP | 9 |
| EPKSCSKYGPTCPPCP | 10 |
| EPKCESKYGPPCPPCP | 11 |
| EPKSCDKTPPCPPCP | 12 |
| EPKSCDKTHPCPPCP | 13 |
| EPKSCDKTPTCPPCP | 14 |
| ESKYGHTCPPCP | 15 |
| ESKYGHPCPPCP | 16 |
| ESKYGPTCPPCP | 17 |

The modified hinge regions described above can be incorporated into a heavy chain constant region, which typically include CH2 and CH3 domains, and which may have an additional hinge segment (e.g., an upper hinge) flanking the designated region, and a CH1 region. Such additional constant region segments present are typically of the same isotype, preferably a human isotype, although can be hybrids of different isotypes. The isotype of such additional human constant regions segments is preferably human IgG1 but can also be human IgG2, IgG3, or IgG4 or hybrids thereof in which domains are of different isotypes.

As used herein, The term “hinge region (or part thereof)” and the term “modified hinge region (or part thereof)”, when referring to a hinge region according to the present invention, are used exchangably as referring to a hinge region with 0 or 1, 2, 3, 4, 5, 6, 7, 8, 9 or 10 substitutions, deletions or internal insertions. A hinge region is considered to be of a designated isotype if it differs from the wilde-type of that isotype by 0 or 1, 2, 3, 4, 5, 6, 7, 8, 9 or 10 substitutions, deletions or internal insertions. As used herein, the expression of a hinge region, a Fab, a Fc fragment or any other component of the polypeptide complex preceded by a designated isotype, like in the case of “an IgG1 hinge region”, means that the hinge region, the Fab, the Fc fragment or any other component is of the designated isotype, but not necessarily the wilde-type.

An interchain bond is formed between one amino acid residue on one single chain of hinge region and another amino acid residue on the other single chain of hinge region. In certain embodiments, the non-native interchain bond can be any bond or interaction that is capable of associating two single chains of hinge region into a dimer. Examples of suitable non-native interchain bond include, a disulphide bond, a hydrogen bond, electrostatic interaction, a salt bridge, or hydrophobic-hydrophilic interaction, a knobs-into-holes or the combination thereof.

The term “dimer” as used herein refers to an associated structure formed by two molecules, such as polypeptides or proteins, via covalent or non-covalent interactions. A homodimer or homodimerization is formed by two identical molecules, and a heterodimer or heterodimerization is formed by two different molecules.

A “disulphide bond” refers to a covalent bond with the structure R—S-S-R′. The amino acid cysteine comprises a thiol group that can form a disulphide bond with a second thiol group, for example from another cysteine residue. The disulphide bond can be formed between the thiol groups of two cysteine residues residing respectively on the two polypeptide chains, thereby forming an interchain bridge or interchain bond.

Electrostatic interaction is non-covalent interaction and is important in protein folding, stability, flexibility and function, including ionic interactions, hydrogen bonding and halogen bonding. Electrostatic interactions can be formed in a polypeptide, for example, between Lys and Asp, between Lys and Glu, between Glu and Arg, or between Glu, Trp on the first chain and Arg, Val or Thr on the second chain.

A salt bridge is close-range electrostatic interactions that mainly arises from the anionic carboxylate of either Asp or Glu and the cationic ammonium from Lys or the guanidinium of Arg, which are spatially proximal pairs of oppositely charged residues in native protein structures. Charged and polar residues in largely hydrophobic interfaces may act as hot spots for binding. Among others, residues with ionizable side chains such as His, Tyr, and Ser can also participate the formation of a salt bridge.

A hydrophobic interaction can be formed between one or more Val, Tyr and Ala on the first chain and one or more Val, Leu, and Trp on the second chain, or His and Ala on the first chain and Thr and Phe on the second chain (see Brinkmann, et al., 2017, Supra).

A hydrogen bond is formed by electrostatic attraction between two polar groups when a hydrogen atom covalently bound to a highly electronegative atom such as nitrogen, oxygen, or fluorine. A hydrogen bond can be formed in a polypeptide between the backbone oxygens (e.g. chalcogen groups) and amide hydrogens (nitrogen group) of two residues, respectively, such as a nitrogen group in Asn and an oxygen group in His, or an oxygen group in Asn and a nitrogen group in Lys. A hydrogen bond is stronger than a Van der Waals interaction, but weaker than covalent or ionic bonds, and is critical in maintaining the secondary structure and tertiary structure. For example, an alpha helix is formed when the spacing of amino acid residues occurs regularly between positions i and i+4, and a beta sheet is a stretch of peptide chain 3-10 amino acids long formed when two peptides joined by at least two or three backbone hydrogen bonds, forming a twisted, pleated sheet.

“Knobs-into-holes” as used herein, refers to an interaction between two polypeptides, where one polypeptide has a protuberance (i.e. “knob”) due to presence of an amino acid residue having a bulky side chain (e.g. tyrosine or tryptophan), and the other polypeptide has a cavity (i.e. “hole”) where a small side chain amino acid residue resides (e.g. alanine or threonine), and the protuberance is positionable in the cavity so as to promote interaction of the two polypeptides to form a heterodimer or a complex. Methods of generating polypeptides with knobs-into-holes are known in the art, e.g., as described in U.S. Pat. No. 5,731,168.

In certain embodiments, the hinge region comprises 1, 2, 3, 4, 5, 6, 7, 8, 9 or 10 interchain bonds. Optionally, at least one of the 1, 2, 3, 4, 5, 6, 7, 8, 9 or 10 interchain bonds are disulphide bonds, hydrogen bonds, electrostatic interaction, salt bridge, or hydrophobic-hydrophilic interaction, or any combination thereof.

Formation of the interchain disulphide bond can be determined by suitable methods known in the art. For example, the expressed protein product can be subject to reduced and non-reduced SDS-PAGE respectively, followed by comparison of the resulting bands to identify potential difference which indicates presence of interchain disulphide bond.

In certain embodiments, the polypeptide complex comprises an antigen-binding fragment Fab of human IgG1 isotype, followed by a modified hinge region of human IgG4 isotype with the S228P mutation for preventing arm exchange, followed by a constant region that comprises the CH2-CH3 domain of IgG (e.g., IgG1, IgG2, IgG3, IgG4, or a combination thereof), wherein the swapping of Fab and hinge subclasses between IgG1 and IgG4 modifies the natural accessibility of reducing agent to the inter-heavy-heavy-chain disulfide bond versus the inter-heavy-light-chain disulfide bond, directing the preferential reduction and payload conjugation onto the inter-heavy-light chain thiols.

In one or more embodiments, exemplary hinge sequences and technical effects thereof are provided in the below Table 2.

TABLE 2
Exemplary hinge sequences and technical effects thereof
of the present disclosure

| mAb ID | Fab Type | Fc Type | Hinge Sequence | D0 | D2 | D4 | D6 | D8 | DAR |
|---|---|---|---|---|---|---|---|---|---|
| 886-2 | IgG4 | IgG1 | DKTHTCPPCP | 4.2 | 10.4 | 58.4 | 24.7 | 2.4 | 4.2 |
| | | | (SEQ No. 1) | | | | | | |
| 886-3 | IgG1 | IgG4 | EPKSCESKYGPPCPPCP | 1.3 | 14.5 | 68.2 | 6.6 | 9.5 | 4.2 |
| | | | (SEQ No. 4) | | | | | | |
| 886-4 | IgG1 | IgG1 | EPKSCESKYGPPCPPCP | 1.5 | 17.8 | 62.0 | 13.9 | 4.7 | 4.1 |
| | | | (SEQ No. 4) | | | | | | |
| 886-5 | IgG4 | IgG4 | DKTHTCPPCP | 5.8 | 17.8 | 65.7 | 2.2 | 8.6 | 3.8 |
| | | | (SEQ No. 1) | | | | | | |
| 886-8 | IgG1 | IgG4 | EPKSCSKYGPPCPPCP | 0.6 | 4.3 | 91.0 | 4.1 | 0.0 | 4.0 |
| | | | (SEQ No. 5) | | | | | | |
| 886-9 | IgG1 | IgG4 | EPKSCKYGPPCPPCP | 1.3 | 5.6 | 87.7 | 4.7 | 0.7 | 4.0 |
| | | | (SEQ No. 6) | | | | | | |
| 886-10 | IgG1 | IgG4 | EPKSCYGPPCPPCP | 0.8 | 10.7 | 82.0 | 5.4 | 1.1 | 3.9 |
| | | | (SEQ No. 7) | | | | | | |
| 886-12 | IgG1 | IgG1 | EPKCESKYGPPCPPCP | 2.1 | 21.3 | 61.1 | 13.6 | 2.0 | 3.8 |
| | | | (SEQ No. 11) | | | | | | |
| 886-13 | IgG1 | IgG1 | EPKSCSKYGPPCPPCP | 1.0 | 6.8 | 85.5 | 6.7 | 0.0 | 4.0 |
| | | | (SEQ No. 5) | | | | | | |
| 886-14 | IgG1 | IgG1 | EPKSCKYGPPCPPCP | 1.2 | 3.8 | 84.9 | 10.2 | 0.0 | 4.1 |
| | | | (SEQ No. 6) | | | | | | |
| 886-15 | IgG1 | IgG1 | EPKSCYGPPCPPCP | 0.8 | 7.4 | 79.5 | 10.7 | 1.6 | 4.1 |
| | | | (SEQ No. 7) | | | | | | |
| 886-16 | IgG1 | IgG4 | EPKSCSKYGPPCPPCP | 0.6 | 5.5 | 87.5 | 5.7 | 0.7 | 4.0 |
| | | | (SEQ No. 5) | | | | | | |
| 886-17 | IgG1 | IgG4 | EPKSCSKYGPPCPPCP | 0.4 | 3.2 | 89.8 | 5.8 | 0.8 | 4.1 |
| | | | (SEQ No. 5) | | | | | | |
| 886-18 | IgG1 | IgG4 | EPKSCSKYGPPCPPCP | 0.3 | 3.0 | 89.0 | 6.6 | 1.1 | 4.1 |
| | | | (SEQ No. 5) | | | | | | |
| 886-19 | IgG1 | IgG1 | EPKSCSKYGPPCPPCP | 1.2 | 7.5 | 85.6 | 5.3 | 0.4 | 3.9 |
| | | | (SEQ No. 5) | | | | | | |
| 886-20 | IgG1 | IgG1 | EPKSCSKYGPPCPPCP | 0.6 | 6.8 | 86.2 | 6.0 | 0.4 | 4.0 |
| | | | (SEQ No. 5) | | | | | | |
| 886-21 | IgG1 | IgG1 | EPKSCSKYGPPCPPCP | 0.7 | 7.1 | 80.2 | 9.8 | 2.2 | 4.1 |
| | | | (SEQ No. 5) | | | | | | |
| 886-22 | IgG1 | IgG1 | EPKSCDKTPPCPPCP | 2.1 | 20.8 | 56.9 | 15.2 | 5.0 | 4.0 |
| | | | (SEQ No. 12) | | | | | | |
| 886-23 | IgG1 | IgG1 | EPKSCDKTHPCPPCP | 4.2 | 20.6 | 44.4 | 24.1 | 6.7 | 4.2 |
| | | | (SEQ No. 13) | | | | | | |
| 886-24 | IgG1 | IgG1 | EPKSCDKTPTCPPCP | 3.6 | 24.1 | 49.9 | 14.8 | 7.6 | 4.0 |
| | | | (SEQ No. 14) | | | | | | |
| 886-25 | IgG4 | IgG4 | ESKYGHTCPPCP | 8.4 | 18.4 | 57.2 | 3.2 | 12.8 | 3.9 |
| | | | (SEQ No. 15) | | | | | | |
| 886-26 | IgG4 | IgG4 | ESKYGHPCPPCP | 7.5 | 14.7 | 59.8 | 3.1 | 14.9 | 4.1 |
| | | | (SEQ No. 16) | | | | | | |
| 886-27 | IgG4 | IgG4 | ESKYGPTCPPCP | 11.0 | 15.7 | 52.0 | 2.4 | 19.1 | 4.1 |
| | | | (SEQ No. 17) | | | | | | |
| 886-28 | IgG4 | IgG4 | EPKSDKTHTCPPCP | 6.8 | 10.6 | 75.1 | 0.0 | 7.4 | 3.8 |
| | | | (SEQ No. 2) | | | | | | |
| 886-29 | IgG4 | IgG4 | EPKDKTHTCPPCP | 5.3 | 8.2 | 78.1 | 0.0 | 8.4 | 4.0 |
| | | | (SEQ No. 3) | | | | | | |
| 886-32 | IgG1 | IgG4 | EPKSCSKYGHTCPPCP | 1.3 | 13.9 | 73.4 | 8.1 | 3.3 | 4.0 |
| | | | (SEQ No. 8) | | | | | | |
| 886-33 | IgG1 | IgG4 | EPKSCSKYGHPCPPCP | 1.5 | 13.4 | 76.5 | 5.0 | 3.6 | 3.9 |
| | | | (SEQ No. 9) | | | | | | |
| 886-34 | IgG1 | IgG4 | EPKSCSKYGPTCPPCP | 1.0 | 11.8 | 82.5 | 4.7 | 0.0 | 3.8 |
| | | | (SEQ No. 10) | | | | | | |

Antibody Drug Conjugate

i. Antibody

Provided herein is a novel antibody comprising, from N-terminus to C-terminus, a Fab domain operably linked to a hinge region, wherein the Fab domain and the hinge region, or part thereof are derived from different IgG isotypes. The antibody comprises at least two heavy chains and two light chains, which have their two heavy chains connected by two disulfide bonds located in the hinge region or part thereof, which is a human IgG1, IgG2, IgG3 or IgG4 hinge region or part thereof.

In another aspect, antibody further comprises a Fc polypeptide which is operably linked to the hinge region or further comprises an additional polypeptide which is operably linked to the hinge region.

In certain embodiments, the Fc polypeptide is a human IgG1, IgG2, IgG3 or IgG4 Fc polypeptide.

In certain embodiments, Fc polypeptide is a human IgG1 or IgG4 Fc polypeptide.

In a further related aspect, the present disclosure includes an antibody drug conjugate comprising a polypeptide complex of the present disclosure.

In context of the present invention, the Fab domain may derive from any antibody, especially those that are clinically relevant. In some embodiments, the Fab domain derives from an antibody that specifically binds to a tumor antigen (TA), such as a tumor specific antigen (TSA) and a tumor-associated antigen (TAA). Examples of the tumor antigen include, but are not limited to, CD20, CD38, CD123, ROR1, ROR2, BCMA, PSMA, SSTR2, SSTR5, CD19, FLT3, CD33, PSCA, ADAM 17, CEA, Her2, EGFR, EGFR-vIII, CD30, FOLR1, GD-2, CA-IX, Trop-2, CD70, CD38, mesothelin, EphA2, CD22, CD79b, GPNMB, CD56, CD138, CD52, CD74, CD30, CD123, RON, and ERBB2. Examples of TA-specific antibodies include, but are not limited to, Trastuzumab (like in Examples 9 and 10 described below) Rituximab (like in Examples 11 and 12), Cetuximab (like in Examples 13 and 14), Bevacizumab, Panitumumab, Alemtuzumab, Matuzuma, Gemtuzumab, Polatuzumab, Inotuzumab, etc.

ii. Drug

The drug (also known as “payload”) used in the present invention is not particularly limited. Drugs for use in the present invention include cytotoxic drugs, particularly those which are used for cancer therapy. Such drugs include, but are not limited to, DNA damaging agents, DNA binding agents, anti-metabolites, enzyme inhibitors such as thymidylate synthase inhibitors and topoisomerase inhibitors, tubulin inhibitors, and toxins (for example, toxins of a bacterial, fungal, plant or animal origin). Specific examples include, for example, taxol, methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, caminomycin, aminopterin, tallysomycin, podophyllotoxin and podophyllotoxin derivatives such as etoposide or etoposide phosphate, vinblastine, vincristine, vindesine, taxanes including taxol, taxotere retinoic acid, butyric acid, N8-acetyl spermidine, camptothecin, calicheamicin, esperamicin, ene-diynes, duocarmycin A, duocarmycin SA, calicheamicin, camptothecin, hemiasterlins, maytansinoids (including DM1, DM2, DM3, DM4), auristatins including monomethylauristatin E (MMAE), monomethylauristatin F (MMAF) and monomethylauristatin D (MMAD). In some embodiments, auristatins, eg., MMAE, are preferred. Drugs can be linked to the linker via any suitable methods known in the art. In some embodiments, the drug is provided for conjugation in the form of a linker-drug intermediate, like in the case of “MC-vc-PAB-MMAE”.

iii. Linker

The drug used in the present invention can be bound to an antibody via a linker. Various linkers for ADCs are known in the art. Linkers useful in the present invention are not particularly limited, as long as it includes a moiety capable reacting a thoil group rendered by an antibody and thereby linking to the antibody. Particularly useful in the present invention are amleimido or haloactyl functionalized linkers. Examples include, but are not limited to -MC-vc-PAB- (“MC”: Maleimide-caproyl; “vc”: the dipeptide of Val-Cit; “PAB”: para-aminobenzyl), -MC-vc-, -MC- and -SMCC-(succinimidyl-4-(N-maleimidomethyl)cyclohexane-1-carboxylate).

In a related aspect, the present disclosure includes a pharmaceutical composition comprising an antibody drug conjugate of the present disclosure and a pharmaceutically acceptable carrier or excipient.

In a still further related aspect, the present disclosure includes a kit comprising a polypeptide complex of the present disclosure or an antibody drug conjugate of the present disclosure or a pharmaceutical composition of the present disclosure.

In another aspect, the present disclosure includes a method of preparing the antibody drug conjugate of the present disclosure, comprising:

providing the polypeptide complex of any one of the present disclosure;

reacting a maleimido or haloacetyl moiety with free thiol group in cysteine residue generated by reduction of interchain disulfide bonds via Michael addition reaction.

In certain embodiments, the free thiol group is generated by partial reduction of interchain disulfide bonds with mild reducing reagent such as TCEP or DTT, preferably the partial reduction is carried out in a buffer with pH range from about 4.0 to 8.0, with reducing agent (e.g., TCEP)/mAb ratio from about 3 to 10, reaction temperature from about 4° C. to 37° C., and reduction time from about 1 hr to 24 hr.

In certain embodiments, the conjugation is carried out in a buffer with pH range from about 4.0 to 8.0, organic additive (e.g., organic solvent or organic co-solvent) from about 0.0% to 20.0% by weight, drug/mAb ratio from about 7 to 20, reaction temperature from about 4° C. to 37° C., and conjugation time from about 1 hr to 4 hr.

In another aspect, the present disclosure includes use of the polypeptide complex of the present disclosure for manufacturing an antibody drug conjugate.

In another aspect, the present disclosure includes a method of treating a condition in a subject in need thereof, comprising administrating to the subject a therapeutically effective amount of the antibody drug conjugate of the present disclosure. The conditions to be treated may include, but are not be limited to, cancers, including solid tumors and hematopoeitic malignancies. Examples of such cancers include, but are not limited to, breast cancers, gastric cancers, lung cancers (e.g., NSCLC), head and neck cancers, colorectal cancers, B cell lymphomas (e.g., non-Hodgkin's lymphoma (NHL)) and leukemias, etc.

The present invention is based at least in part on employing two immunoglobulin heavy chain hinge region sequences. By swapping the hinge region of IgG1 and IgG4 immunoglobulin at their natural structural positions on the C-terminus of Fab, such difference results in an improved payload-antibody ratio (PAR) during bio-conjugation, because it leads to a differential accessibility of reducing agent to the inter-chain disulfide bond.

The present disclosure describes a type of antibody constituted with an engineered hinge region, a Fab domain and a Fc domain. Peptides of the engineered hinge region are constituted with natural amino acids, including cysteine residues for the formation of two disulfide bonds between two heavy chains. Specifically, sequences of hinge region in IgG1 and IgG4 are truncated and combined to get the hinge region of engineered antibody, with two disulfide bonds retained. Fab domain of this engineered antibody can be of IgG1 isotype or IgG4 isotype, any mutation, including but not limited to Cysteine, non-natural amino acid or peptide extension or insertion, can be applied in the Fab domain. Also, Fc domain can be of IgG1 isotype or IgG4 isotype, with or without mutations.

The engineered antibody according to the present invention may be used for bio-conjugation at cysteine residue after reduction of disulfide bonds by mild reduction reagents. Engineered peptide alters the reducing property of disulfide bonds in hinge region, results in selected reduction of disulfide bonds when partially reduced by mild reductants such as TCEP or DTT. Application of this partial reduced antibody in conjugation results in conjugate with four linker-payloads attached at specific sites as main product. According to the present invention, linker-drug can be attached to either Fab region or hinge region according to different combination of Fab domain, hinge region and Fc domain. In some of the selected examples, conjugate with four linker-payloads attached at specific sites occupies over 90% in the product mixture.

The present disclosure also describes the method of using the engineered antibodies in bio-conjugation reaction. The overall conjugation reaction is comprised by two steps: partially reaction and conjugation. Reductant type, reductant/mAb ratio, buffer component and pH, reaction temperature and time may be influential to partial and site-specific reduction. Conjugation condition is mostly the same as conventional condition generally used currently, and is depended on property of linker-payload needs to be attached on antibody. Ideally, conjugation is performed in reduction buffer with organic solvent as additive to help the dissolve of linker-payload.

In one aspect, an antigen-binding immunoglobulin G is provided, comprising, from N-terminus to C-terminus, an antigen-binding fragment Fab of human IgG4 isoype, followed by a modified hinge region of human IgG1 isotype, followed by a constant region that comprises the CH2-CH3 domain of IgG (e.g., IgG1, IgG2, IgG3, IgG4, or a combination thereof); wherein the swapping of Fab and hinge subclasses between IgG4 and IgG1 modifies the natural accessibility of reducing agent to the inter-heavy-heavy-chain disulfide bond versus the inter-heavy-light chain disulfide bond, directing the preferential reduction and payload conjugation onto the inter-heavy-heavy chain thiols.

In another aspect, an antigen-binding immunoglobulin G is provided, comprising, from N-terminus to C-terminus, an antigen-binding fragment Fab of human IgG1 isotype, followed by a modified hinge region of human IgG4 isotype with the S228P mutation for preventing arm exchange, followed by a constant region that comprises the CH2-CH3 domain of IgG (e.g., IgG1, IgG2, IgG3, IgG4, or a combination thereof); wherein the swapping of Fab and hinge subclasses between IgG1 and IgG4 modifies the natural accessibility of reducing agent to the inter-heavy-heavy-chain disulfide bond versus the inter-heavy-light-chain disulfide bond, directing the preferential reduction and payload conjugation onto the inter-heavy-light chain thiols.

In one embodiment, the CH1 and Hinge junction of the swapped hinge comprises a 1, 2, or 3-amino acid deletion in the upper hinge (e.g., 216-223 by EU numbering). In specific embodiments, the hinge fragment is selected from SEQ ID NOs: 1-17.

The present disclosure also describes conjugation methods accomplished using a maleimido or haloacetyl moiety which can react with thiol group in cysteine residue generated by reduction of interchain disulfide bonds via Michael addition reaction.

In some embodiments, generation of free thiol group can be accomplished by partial reduction of interchain disulfide bonds with mild reducing reagent such as TCEP or DTT. Partial reduction of disulfide bonds can be performed in a buffer with pH range from about 4.0 to 8.0, with reducing agent (e.g., TCEP)/mAb ratio from about 3 to 10, reaction temperature from about 4° C. to 37° C., and reduction time from about 1 hr to 24 hr.

In some embodiments, conjugation of partial reduced antibody with maleimido functionalized linker-payload can be performed in a buffer with pH range from about 4.0 to 8.0, organic additive (e.g., organic solvent or organic co-solvent) from about 0.0% to 20.0%, drug/mAb ratio from about 7 to 20, reaction temperature from about 4° C. to 37° C., and conjugation time from about 1 hr to 4 hr.

### Abbreviation

ADC: Antibody-drug conjugate
CH: Constant domain on heavy chain
CMC: Chemistry, Manufacturing and Controls
DAR: Drug-antibody ratio
DMA: N,N′-Dimethylacetamide
DTT: 1,4-Dithiothreitol
EGFR: Epidermal growth factor receptor
Fab: Antigen-binding fragment
Fc: Fragment, crystallizable
FDA: Food and Drug Administration
FGE: Formylglycine-generating enzyme
HIC: Hydrophobic interaction chromatography
HPLC: High performance liquid chromatography
IC50: The half maximal inhibitory concentration
IgG: Immunoglobulin G
MC: Maleimide-caproyl
MED: Minimum Effect Dose
MMAE: Monomethyl auristatin E
MTD: Maximum Tolerance Dose
MWCO: Molecular weight cut-off
NaCl: Sodium Chloride
NNAA: Non-natural amino acid
mTG: Microbial transglutaminase
PAB: para-aminobenzyl
PAR: Payload-antibody ratio
RP: Reverse phase
SEC: Size exclusion chromatography
TCEP: Tris(2-carboxyethyl)phosphine
VH: Variable domain on heavy chain
eq: Molar ratio of reducing agent/mAb

### Method

Antibody Preparation

All antibody molecules described in this document were codon optimized for Cricetulus griseus, synthesized, cloned into proprietary production vectors, and then maxi-prepared from TOP10 E. coli cells following standard molecular biology procedures.

CHO K1 host cells were seeded at 2-4E5 cells/mL in CD CHO medium 72 hours before transfection. The host cells were counted for cell density using Vi-CELL, centrifuged at 290 g for 7 min and then resuspended in pre-warmed fresh CD CHO medium prior to transfection. The re-suspended host cells were incubated in a Kuhner shaker (36.5° C., 75% humidity, 6% CO₂, 120 rpm) before use.

A total 4 mg of plasmids encoding the antibody of interest were added into the re-suspended host cells, followed by 12 mg polyetherimide. The transfected cultures were incubated in a Kuhner shaker at 36.5° C., 75% humidity, 6% CO₂, 120 rpm for 4 hours. After proprietary supplements were added, the transfected cultures were then incubated in a Kuhner shaker at 31° C., 75% humidity, 6% CO₂, 120 rpm for 9-10 days.

On the harvest day, transfected cultures were clarified by primary centrifugation at 1,000 g for 10 min, and secondary centrifugation at 10,000 g for 40 min, followed by sterile filtration through 0.22 μm filter. The supernatants were measured for titers and purified by ProA chromatography. The ProA eluate were neutralized by adding 1-2% neutralization buffer (1 M Tris-HCl, pH 9.0) and formulated in 20 mM Histidine-Acetate buffer, pH 5.5.

All proteins were subjected to quality control tests before conjugation, including reducing and non-reducing SDS-PAGE, SEC-HPLC, endotoxin level detection by LAL gel clot assay and molecular identification by mass spectrometry.

HIC-HPLC

HPLC parameters

| Equipment | Agilent 1260 series HPLC | | | |
|---|---|---|---|---|
| Column | TSKgel Butyl-NPR, (2.5) 4.6*35, 0014947 | | | |
| Column Temp. | 25° C. | | | |
| Mobile phase | A: 1.5M (NH₄)₂SO₄, 50 mM K₂HPO₄, PH 7.0 | | | |
| | B: 50 mM K₂HPO₄, 25% IPA, PH 7.0 | | | |
| Flow rate | 0.5-0.8 mL/min | | | |
| Sampler Temp. | 23° C. | | | |
| Injection volume | 40-50 μg | | | |
| Detection wavelength | 280 nm | | | |
| Gradient | Time (min) | A % | B % | Flow (ml/min) |
| | 0 | 100 | 0 | 0.5 |
| | 12 | 0 | 100 | 0.8 |
| | 17 | 0 | 100 | 0.8 |
| | 18 | 100 | 0 | 0.8 |
| | 30 | 100 | 0 | 0.8 |

SEC-HPLC

HPLC parameters

| Equipment | Agilent 1260 series HPLC | | |
|---|---|---|---|
| Column | TSKgel G3000SWXL ((5) 7.8*300) | | |
| Column Temp. | 25° C. | | |
| Mobile phase | A: 200 mM KPi, 250 mM KCl, 15% IPA, PH 7.0 | | |
| Flow rate | 0.75 mL/min | | |
| Sampler Temp. | 4° C. | | |
| Injection volume | 50 ug | | |
| Detection wavelength | 280 nm, 252 nm, 248 nm | | |
| Gradient | Time (min) | A % | B % |
| | 0 | 100 | 0 |
| | 18 | 100 | 0 |

RP-HPLC for Drug Loading

Procedure: mix 20 ul of ADC sample with 75ul 8M Guanidine-HCl and 5 ul Tris-HCl, pH 8.0. Then add 1 ul 0.5M TCEP solution into the mixture. The reaction was performed at 37° C. for 30 min and then tested with RP-HPLC for drug loading on antibody.

HPLC parameters

| Equipment | Agilent 1260 series HPLC | | |
|---|---|---|---|
| Column | Agilent, PLRP-S 2.1 × 150 mm, 8 μm or equivalent | | |
| Column oven temp. | 80° C. | | |
| Mobile phase | A: 0.05% (v/v) TFA in H₂O | | |
| | B: 0.05% (v/v) TFA in ACN | | |
| Flow rate | 0.8 mL/min | | |
| Injection volume | 20 μl | | |
| Detection | 280 nm | | |
| wavelength | | | |
| Gradient Program | Time (min) | % A | % B |
| | 0 | 75 | 25 |
| | 3 | 75 | 25 |
| | 28 | 50 | 50 |
| | 30 | 5 | 95 |
| | 32 | 5 | 95 |
| | 33 | 75 | 25 |
| | 40 | 75 | 25 |

RP-HLPC for Free Drug Determination

Procedure: 85 ul ADC solution was mixed with 15 ul DMA and then protein was precipitated with 100 ul precipitation buffer (37.5% v/v of Methanol in Acetonitrile, saturated with NaCl) and vortex at 1400 rpm for 10 min at 22° C.

Sample was centrifuged at 16000 rpf for 10 min. The supernatant was taken for RP-HPLC assay together with standard sample for free drug determination.

HPLC parameters

| Equipment | Agilent 1260 series HPLC | | |
|---|---|---|---|
| Column | Agilent poroshell 120SB-C18 2.7 um, 4.6*100 mm | | |
| Column Temp. | 40° C. | | |
| Mobile phase | A: 0.05% (v/v) TFA in H₂O | | |
| | B: 0.05% (v/v) TFA in ACN | | |
| Flow rate | 0.6 ml/min | | |
| Sampler Temp. | 10° C. | | |
| Injection volume | 40 ul | | |
| Detection wavelength | 248 nm | | |
| Gradient | Time (min) | A% | B% |
| | 0 | 55 | 45 |
| | 1 | 55 | 45 |
| | 11 | 30 | 70 |
| | 12.5 | 5 | 95 |
| | 14 | 5 | 95 |
| | 14.1 | 55 | 45 |
| | 16 | 55 | 45 |

### EXAMPLES

The following Examples illustrate the invention.

### Example 1

General Conjugation Procedure

To an antibody solution concentration 1 mg/ml to 20 mg/ml in a buffer with pH 4.0-8.0, such as Histidine-acetate, 1 to 20 eq (e.g., in some embodiments, 3-10eq) of reducing reagent such as TCEP or DTT was added. The reduction was performed at 4-37° C. for 0.5 hr to 24 hr with gentle shaking or stirring. Without purification, organic co-solvent such as DMA was added to the partial reduced antibody to a concentration of 0% to 20%, with 7-20eq of Maleimido or Haloacetyl functionalized linker-payload. The conjugation was performed at 4-37° C. for 0.5 hr to 4 hr with gentle shaking or stirring. Final conjugated product was characterized with UV-vis for concentration, HIC-HPLC for conjugate distribution and DAR, RP-HPLC for drug loading on light chain and heave chain as well as free drug residue, SEC-HPLC for aggregation and purity, and kinetic turbidimetric for Endotoxin level.

All antibody molecules described in this document were codon optimized for Cricetulus griseus, synthesized, cloned into proprietary production vectors, and then maxi-prepared from TOP10 E. coli cells following standard molecular biology procedures.

CHO K1 host cells were seeded at 2-4E5 cells/mL in CD CHO medium 72 hours before transfection. The host cells were counted for cell density using Vi-CELL, centrifuged at 290 g for 7 min and then resuspended in pre-warmed fresh CD CHO medium prior to transfection. The re-suspended host cells were incubated in a Kuhner shaker (36.5° C., 75% humidity, 6% CO₂, 120 rpm) before use.

A total 4 mg of plasmids encoding the antibody of interest were added into the re-suspended host cells, followed by 12 mg polyetherimide. The transfected cultures were incubated in a Kuhner shaker at 36.5° C., 75% humidity, 6% CO₂, 120 rpm for 4 hours. After proprietary supplements were added, the transfected cultures were then incubated in a Kuhner shaker at 31° C., 75% humidity, 6% CO₂, 120 rpm for 9-10 days.

On the harvest day, transfected cultures were clarified by primary centrifugation at 1,000 g for 10 min, and secondary centrifugation at 10,000 g for 40 min, followed by sterile filtration through 0.22 μm filter. The supernatants were measured for titers and purified by ProA chromatography. The ProA eluate were neutralized by adding 1-2% neutralization buffer (1 M Tris-HCl, pH 9.0) and formulated in 20 mM Histidine-Acetate buffer, pH 5.5.

All proteins were subjected to quality control tests before conjugation, including reducing and non-reducing SDS-PAGE, SEC-HPLC, endotoxin level detection by LAL gel clot assay and molecular identification by mass spectrometry.

The IgG1 antibody and the IgG4 antibody, without isotype swapping, were prepared as described above. The IgG1 antibody has a hinge sequence of EPKSCDKTHTCPPCP (SEQ ID NO: 18) and the IgG4 antibody a hinge sequence of ESKYGPPCPPCP (SEQ ID NO: 19). The antibodies were dissolved in 50 mM PB, 50 mM NaCl, 2 mM EDTA, PH 7.0 to a concentration of 8.0 mg/ml, 50 mM PB and 50 mM NaCl, 2 mM EDTA, PH 6.5 to a concentration of 8.0 mg/ml, respectively. For the IgG1 antibody, 2.7 eq of TCEP was added and the mixture was incubated at 37° C. for 2 hrs. For the IgG4 antibody, 4.1 eq of TCEP was added and the mixture was incubated at 37° C. for 24 hrs.

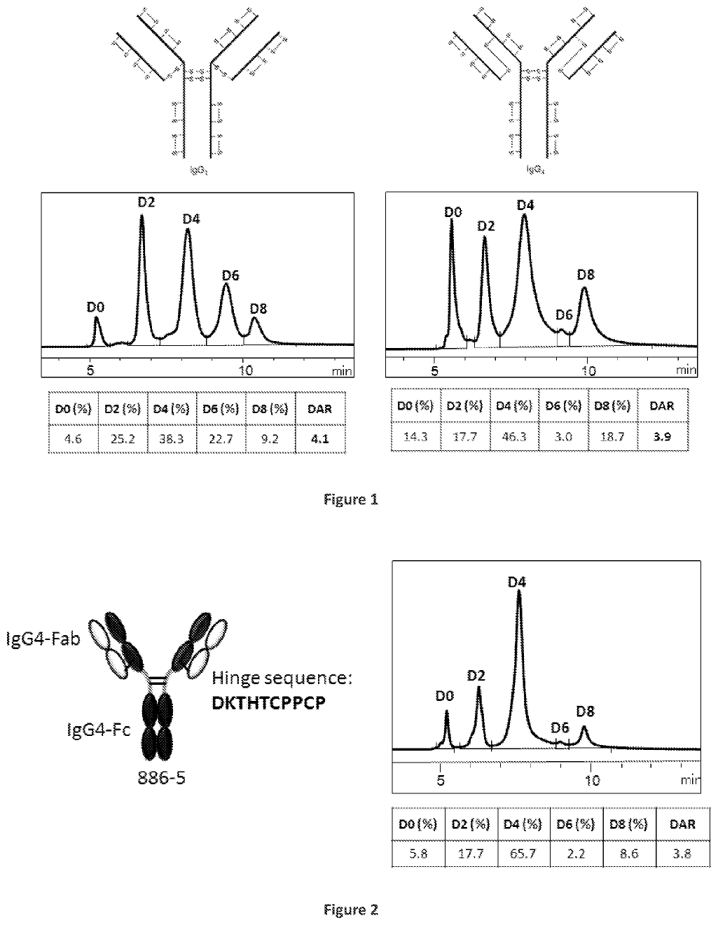

Then, for each mixture, DMA was added to the reduced antibody to a concentration of 10%, followed by 7 eq (for IgG1) and 9 eq (for IgG4) of MC-vc-PAB-MMAE, respectively. Conjugation reaction was performed at 4° C. for 1 hr. The conjugated products were purified with 40KD MWCO desalting column and stored in 20 mM Histidine-acetate pH 5.5. Final products were characterized with HIC-HPLC for DAR and drug distribution determination (FIG. 1).

### Example 2

Antibody 886-5 (IgG4-Fab, IgG4-Fc, Hinge sequence is DKTHTCPPCP (SEQ ID NO: 1)) was dissolved in 20 mM Histidine-acetate, 150 mM NaCl, pH 6.0 to a concentration of 7.0 mg/ml. 3.5eq of TCEP was added to the antibody solution and the mixture was incubated at 15° C. for 18 hr. Then DMA was added to the reduced antibody to a concentration of 10%, followed by 7eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination (FIG. 2).

### Example 3

Antibody 886-5 (IgG4-Fab, IgG4-Fc, Hinge sequence is DKTHTCPPCP (SEQ ID NO: 1)) was dissolved in 20 mM Histidine-acetate pH 6.0 to a concentration of 7.0 mg/ml. 3.3eq of TCEP was added to the antibody solution and the mixture was incubated at 15° C. for 18 hr. Then DMA was added to the reduced antibody to a concentration of 10%, followed by 7eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination. Results were reported below:

| | TCEP | | | | | | |
|---|---|---|---|---|---|---|---|
| mAb | ratio/T | D0 | D2 | D4 | D6 | D8 | DAR |
| 886-5 | 3.3/15° C. | 6.9 | 18.6 | 57.6 | 3.5 | 13.4 | 4.0 |

### Example 4

Antibody 886-5 (IgG4-Fab, IgG4-Fc, Hinge sequence is DKTHTCPPCP (SEQ ID NO: 1)) was dissolved in 20 mM HEPES pH 8.0 to a concentration of 5.7 mg/ml. 2.6eq of TCEP was added to the antibody solution and the mixture was incubated at 15° C. for 16 hr. Then DMA was added to the reduced antibody to a concentration of 10%, followed by 7eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination. Results were reported below:

| | TCEP | | | | | | |
|---|---|---|---|---|---|---|---|
| mAb | ratio/T | D0 | D2 | D4 | D6 | D8 | DAR |
| 886-5 | 2.6/15° C. | 7.5 | 19.2 | 57.8 | 1.2 | 14.3 | 3.9 |

### Example 5

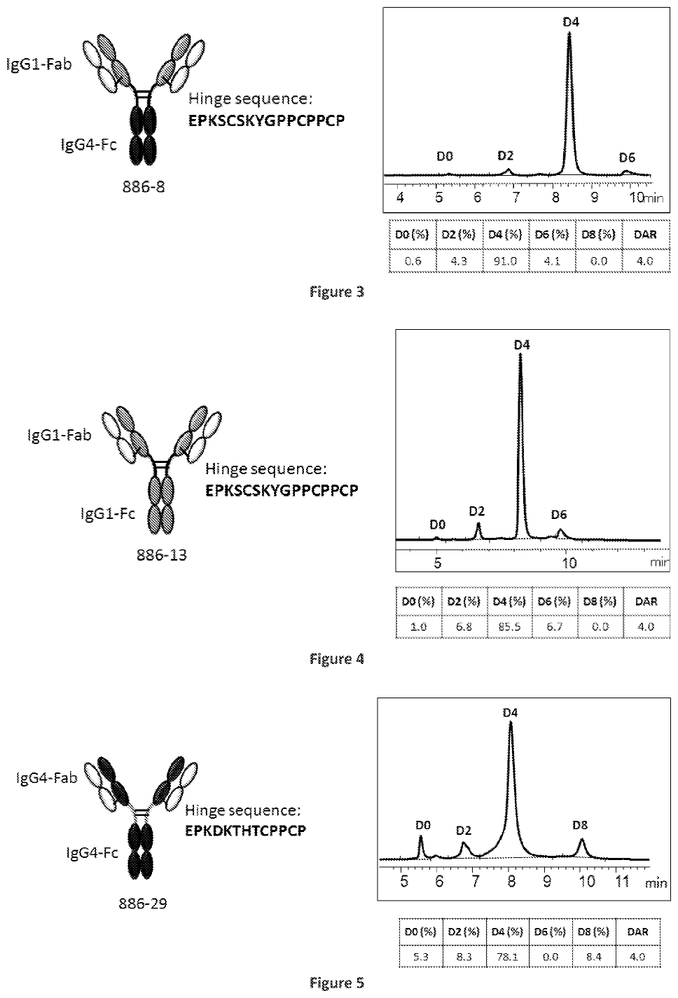

Antibody 886-8 (IgG1-Fab, IgG4-Fc, Hinge sequence is EPKSCSKYGPPCPPCP (SEQ ID NO: 5)) was dissolved in 20 mM Histidine-acetate pH 5.5 to a concentration of 4 mg/ml. 7eq of TCEP was added to the antibody solution and the mixture was incubated at 4° C. for 3 hr. Then DMA was added to the reduced antibody to a concentration of 10%, followed by 12eq of MC-vc-PAB-MMAE. Conjugation reaction was performed at 22° C. for 0.5 hr. The conjugated product was purified with 40KD MWCO desalting column and stored in 20 mM Histidine-acetate pH 5.5. Final product was characterized with HIC-HPLC for DAR and drug distribution determination (FIG. 3).

### Example 6

Antibody 886-13 (IgG1-Fab, IgG1-Fc, Hinge sequence is EPKSCSKYGPPCPPCP (SEQ ID NO: 5)) was dissolved in 20 mM Histidine-acetate pH 5.5 to a concentration of 4.0 mg/ml. 4.4eq of TCEP was added to the antibody solution and the mixture was incubated at 10° C. for 3 hr. Then DMA was added to the reduced antibody to a concentration of 10%, followed by 10eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination (FIG. 4).

### Example 7

Antibody 886-29 (IgG4-Fab, IgG4-Fc, Hinge sequence is EPKDKTHTCPPCP (SEQ ID NO: 3)) was dissolved in 20 mM Histidine-acetate pH 5.5 to a concentration of 7.8 mg/ml. 6.0eq of TCEP was added to the antibody solution and the mixture was incubated at 37° C. for 2 hr. Then DMA was added to the reduced antibody to a concentration of 10%, followed by 7eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination (FIG. 5).

### Example 8

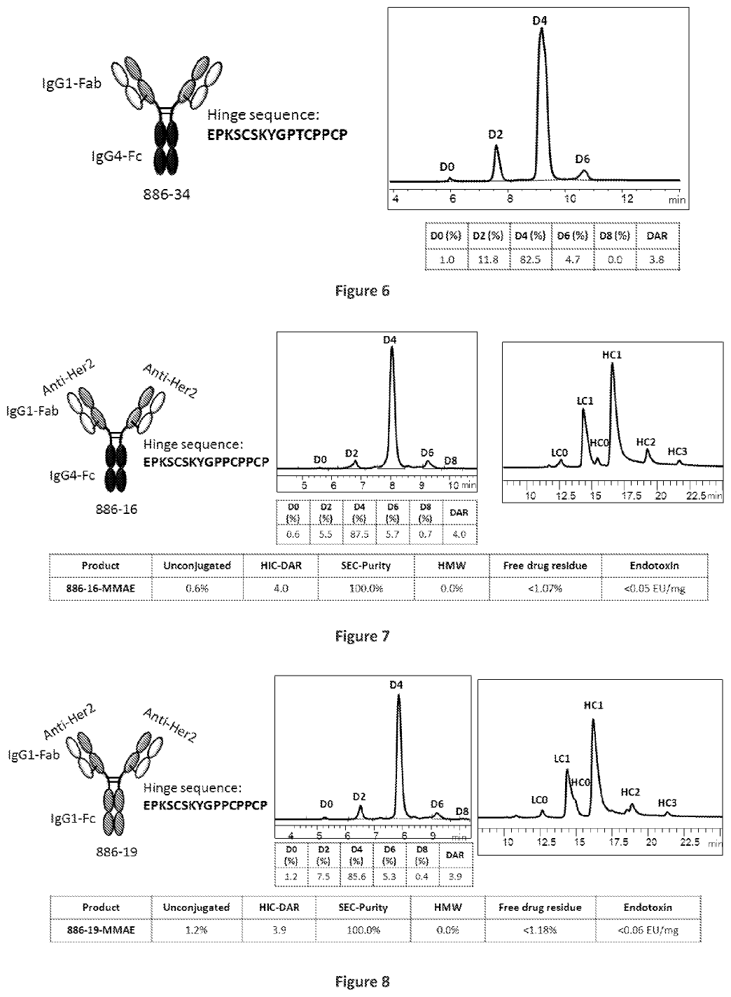

Antibody 886-34 (IgG1-Fab, IgG4-Fc, Hinge sequence is EPKSCSKYGPTCPPCP (SEQ ID NO: 10)) was dissolved in 20 mM Histidine-acetate pH 5.5 to a concentration of 6.2 mg/ml. 5.0eq of TCEP was added to the antibody solution and the mixture was incubated at 4° C. for 2 hr. Then DMA was added to the reduced antibody to a concentration of 10%, followed by 7eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination (FIG. 6).

### Example 9

Anti-Her2 antibody 886-16 (IgG1-Fab, IgG4-Fc; Hinge sequence is EPKSCSKYGPPCPPCP (SEQ ID NO: 5); Sequence of light chain (LC): SEQ ID NO: 20, Sequence of heavy chain (HC): SEQ ID NO: 21) was dissolved in 20 mM Histidine-acetate pH 5.5 to a concentration of 9.2 mg/ml. 5eq of TCEP was added to the antibody solution and the mixture was incubated at 4° C. for 2 hr. Then DMA was added to the reduced antibody to a concentration of 10%, followed by 7eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination, SEC-HPLC for purity and aggregation level test, RP-HPLC for drug loading test, RP-HPLC for free drug residue and kinetic turbidimetric for Endotoxin level (FIG. 7).

### Example 10

Anti-Her2 antibody 886-19 (IgG1-Fab, IgG1-Fc; Hinge sequence is EPKSCSKYGPPCPPCP (SEQ ID NO: 5); LC sequence: SEQ ID NO: 26, HC sequence: SEQ ID NO: 27) was dissolved in 20 mM Histidine-acetate pH 5.5 to a concentration of 7.7 mg/ml. 3eq of TCEP was added to the antibody solution and the mixture was incubated at 4° C. for 3 hr. Then DMA was added to the reduced antibody to a concentration of 10%, followed by 7eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination, SEC-HPLC for purity and aggregation level test, RP-HPLC for drug loading test, RP-HPLC for free drug residue and kinetic turbidimetric for Endotoxin level (FIG. 8).

### Example 11

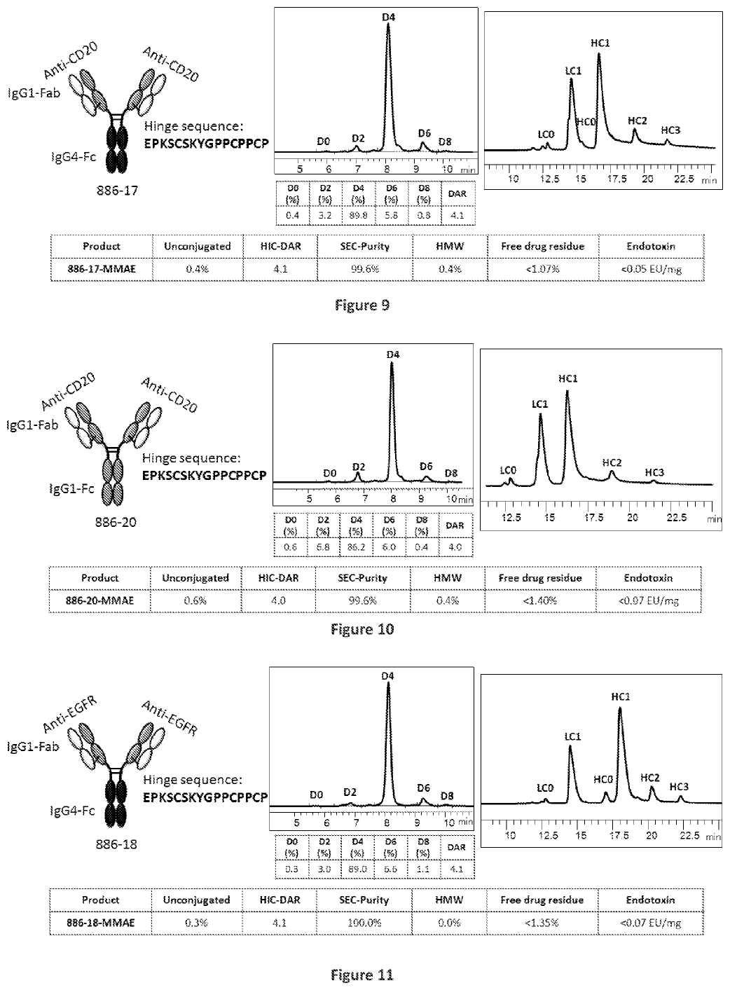

Anti-CD20 antibody 886-17 (IgG1-Fab, IgG4-Fc; Hinge sequence is EPKSCSKYGPPCPPCP (SEQ ID NO: 5); LC sequence: SEQ ID NO: 22, HC sequence: SEQ ID NO: 23) was dissolved in 20 mM Histidine-acetate pH 5.5 to a concentration of 8.9 mg/ml. 5eq of TCEP was added to the antibody solution and the mixture was incubated at 4° C. for 2 hr. Then DMA was added to the reduced antibody to a concentration of 10%, followed by 7eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination, SEC-HPLC for purity and aggregation level test, RP-HPLC for drug loading test, RP-HPLC for free drug residue and kinetic turbidimetric for Endotoxin level (FIG. 9).

### Example 12

Anti-CD20 antibody 886-20 (IgG1-Fab, IgG1-Fc; Hinge sequence is EPKSCSKYGPPCPPCP (SEQ ID NO: 5); LC sequence: SEQ ID NO: 28, HC sequence: SEQ ID NO: 29) was dissolved in 20 mM Histidine-acetate pH 5.5 to a concentration of 7.2 mg/ml. 3eq of TCEP was added to the antibody solution and the mixture was incubated at 4° C. for 3 hr. Then DMA was added to the reduced antibody to a concentration of 10%, followed by 7eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination, SEC-HPLC for purity and aggregation level test, RP-HPLC for drug loading test, RP-HPLC for free drug residue and kinetic turbidimetric for Endotoxin level (FIG. 10).

### Example 13

Anti-EGFR antibody 886-18 (IgG1-Fab, IgG4-Fc; Hinge sequence is EPKSCSKYGPPCPPCP (SEQ ID NO: 5); LC sequence: SEQ ID NO: 24, HC sequence: SEQ ID NO: 25) was dissolved in 20 mM Histidine-acetate pH 5.5 to a concentration of 7.5 mg/ml. 5eq of TCEP was added to the antibody solution and the mixture was incubated at 4° C. for 2 hr. Then DMA was added to the reduced antibody to a concentration of 10%, followed by 7eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination, SEC-HPLC for purity and aggregation level test, RP-HPLC for drug loading test, RP-HPLC for free drug residue and kinetic turbidimetric for Endotoxin level (FIG. 11).

### Example 14

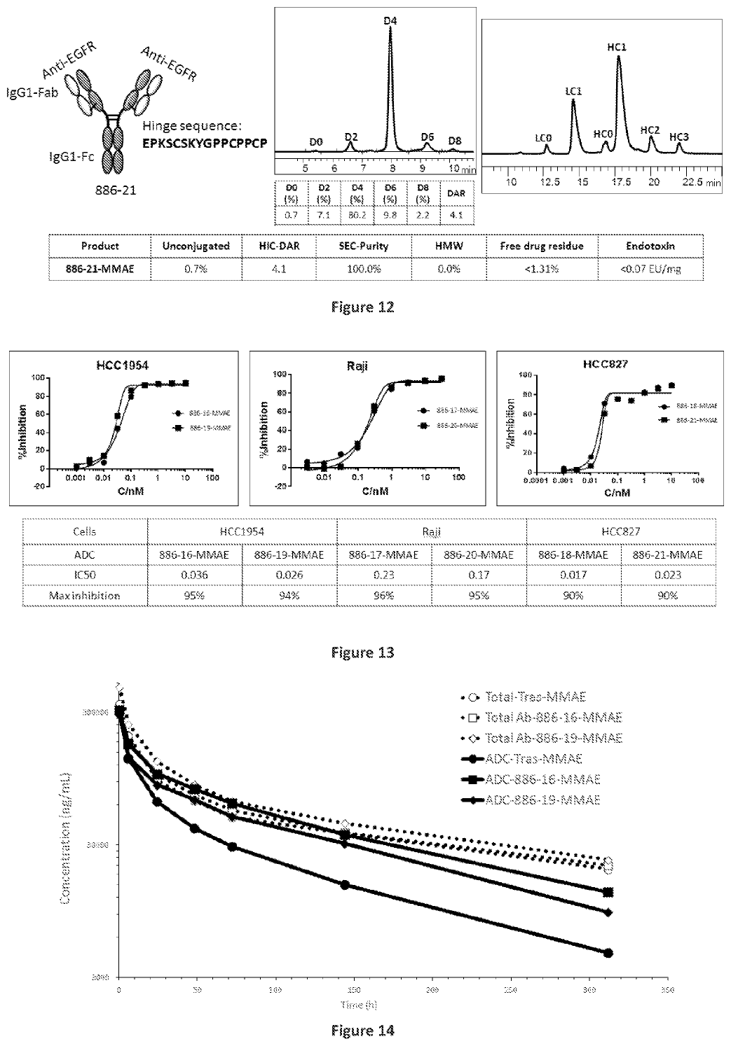

Anti-EGFR antibody 886-21 (IgG1-Fab, IgG1-Fc; Hinge sequence is EPKSCSKYGPPCPPCP (SEQ ID NO: 5); LC sequence: SEQ ID NO: 30, HC sequence: SEQ ID NO: 31) was dissolved in 20 mM Histidine-acetate pH 5.5 to a concentration of 7.0 mg/ml. 3eq of TCEP was added to the antibody solution and the mixture was incubated at 4° C. for 3 hr. Then DMA was added to the reduced antibody to a concentration of 10%, followed by 7eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination, SEC-HPLC for purity and aggregation level test, RP-HPLC for drug loading test, RP-HPLC for free drug residue and kinetic turbidimetric for Endotoxin level (FIG. 12).

### Example 15

Antibody 886-28 (IgG4-Fab, IgG4-Fc, Hinge sequence is EPKSDKTHTCPPCP (SEQ ID NO: 2)) was dissolved in 20 mM Histidine-acetate pH 5.5 to a concentration of 7.2 mg/ml. 6.0eq of TCEP was added to the antibody solution and the mixture was incubated at 37° C. for 2 hr. Then DMA was added to the reduced antibody to a concentration of 10%, followed by 7eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination. Result was reported below:

| | TCEP | | | | | | |
|---|---|---|---|---|---|---|---|
| mAb | ratio/T | D0 | D2 | D4 | D6 | D8 | DAR |
| 886-28 | 6/37° C. | 6.8 | 10.6 | 75.1 | 0.0 | 7.4 | 3.8 |

### Example 16

Antibody 886-22 (IgG1-Fab, IgG1-Fc, hinge sequence is EPKSCDKTPPCPPCP (SEQ ID NO: 12)), antibody 886-23 (IgG1-Fab, IgG1-Fc, hinge sequence is EPKSCDKTHPCPPCP (SEQ ID NO: 13)) and antibody 886-24 (IgG1-Fab, IgG1-Fc, hinge sequence is EPKSCDKTPTCPPCP (SEQ ID NO: 14)) were dissolved in 20 mM Histidine-acetate pH 5.5 to a concentration of 6.9 mg/ml, 7.8 mg/ml, 7.8 mg/ml, respectively. To each of the antibody solutions, TCEP was added at the TCEP/antibody ratio of 5.2, 3.2, 4.6, respectively. The mixtures were incubated at the temperature (T) as specified in the Table below for 2 hr. Then DMA was added to the reduced antibody to a concentration of 10%, followed by 7eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination. Results were reported below:

| | TCEP | | | | | | |
|---|---|---|---|---|---|---|---|
| mAb | ratio/T | D0 | D2 | D4 | D6 | D8 | DAR |
| 886-22 | 5.2/37° C. | 2.1 | 20.8 | 56.9 | 15.2 | 5.0 | 4.0 |
| 886-23 | 3.2/37° C. | 4.2 | 20.6 | 44.4 | 24.1 | 6.7 | 4.2 |
| 886-24 | 4.6/37° C. | 3.6 | 24.1 | 49.9 | 14.8 | 7.6 | 4.0 |

### Example 17

Antibody 886-25 (IgG4-Fab, IgG4-Fc, hinge sequence is ESKYGHTCPPCP (SEQ ID NO: 15)), antibody 886-26 (IgG4-Fab, IgG4-Fc, hinge sequence is ESKYGHPCPPCP (SEQ ID NO: 16)) and antibody 886-27 (IgG4-Fab, IgG4-Fc, hinge sequence is ESKYGPTCPPCP (SEQ ID NO: 17) were dissolved in 20 mM Histidine-acetate pH 5.5 to a concentration of 4.8 mg/ml, 7.2 mg/ml, 7.5 mg/ml, respectively. To each of the antibody solutions, TCEP was added at the TCEP/antibody ratio of 3.5, 4.0, 5.5, respectively. The mixtures were incubated at the temperature (T) as specified in the Table below for 16 hr. Then DMA was added to the reduced antibody to a concentration of 10%, followed by 7eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination. Results were reported below:

| | TCEP | | | | | | |
|---|---|---|---|---|---|---|---|
| mAb | ratio/T | D0 | D2 | D4 | D6 | D8 | DAR |
| 886-25 | 3.5/37° C. | 8.4 | 18.4 | 57.2 | 3.2 | 12.8 | 3.9 |
| 886-26 | 4.0/37° C. | 7.5 | 14.7 | 59.8 | 3.1 | 14.9 | 4.1 |
| 886-27 | 5.5/37° C. | 11.0 | 15.7 | 52.0 | 2.4 | 19.1 | 4.1 |

### Example 18

886-32 (IgG1-Fab, IgG4-Fc, Hinge sequence is EPKSCSKYGHTCPPCP (SEQ ID NO: 8)) and 886-33 (IgG1-Fab, IgG4-Fc, Hinge sequence is EPKSCSKYGHPCPPCP (SEQ ID NO: 9)) were dissolved in 20 mM Histidine-acetate pH 5.5 to a concentration of 9.5 mg/ml and 7.5 mg/ml, respectively. To each of the antibody solutions, TCEP was added at the TCEP/antibody ratio of 1.5 and 2.0, respectively. The mixtures were incubated at the temperature (T) as specified in the Table below for 2 hr. Then DMA was added to the reduced antibody to a concentration of 10%, followed by 7eq of MC-vc-PAB-MMAE. Conjugation reaction was performed at 4° C. for 1 hr. The conjugated product was purified with 40KD MWCO desalting column and stored in 20 mM Histidine-acetate pH 5.5. Final product was characterized with HIC-HPLC for DAR and drug distribution determination. Results were reported below:

| | TCEP | | | | | | |
|---|---|---|---|---|---|---|---|
| mAb | ratio/T | D0 | D2 | D4 | D6 | D8 | DAR |
| 886-32 | 1.5/4° C. | 1.3 | 13.9 | 73.4 | 8.1 | 3.3 | 4.0 |
| 886-33 | 2.0/4° C. | 1.5 | 13.4 | 76.5 | 5.0 | 3.6 | 3.9 |

### Example 19

In vitro cytotoxicity experiment: The antibody-drug conjugates targeting Her2, CD20 and EGFR were tested for cytotoxicity on HCC1954 cells, Raji cells and HCC827 cells, respectively. All three cell lines were cultured in RPMI-1640 medium supplemented with 10% fetal bovine serum. HCC1954 cells were plated in 96-well-plate at 4000 cells/well, Raji cells were plated in 96-well-plate at 10000 cells/well, and HCC827 cells were plated in 96-well-plate at 3000 cells/well. Raji cells were treated with ADCs upon completion of cells plating, HCC1954 and HCC827 cells were treated with ADCs 24 hr after cells were plated. Viability of Raji cells was analyzed after 4 days treatment with ADCs at 37° C. and Viability of HCC1954 and HCC827 cells were analyzed after 5 days treatment with ADCs at 37° C. Percentage of inhibition and maximum Percentage of inhibition were calculated (FIG. 13).

### Example 20

Male SD rats were fed in house to body weights approximately 330 g at dosing. Following a single dose of 10 mg/kg intravenous administration at duplicates for Trastuzumab-MMAE, 886-16-MMAE and 886-19-MMAE, rat plasmas were taken at 5 min, 6 h, 24 h, 48 h, 72 h, 144 h and 312 h, respectively.

Concentrations of total antibody in plasmas collected at different timepoint were determined by ELISA method: 96-well-plate was coated with Recombinant Human ErbB2 (HER2) at lug/mL for 24 h at 4° C. Then plate was blocked by 2% BSA dissolved in PBS, pH7.2 for 1 h at 37° C. After washed with wash buffer (0.05% Tween 20 in PBS pH7.2) for three times, samples with different dilution was incubated with the coated 96-well-plate, concentration of plasma was normalized to 0.1%. A standard curve prepared by ADC diluted in 0.1% plasma was also prepared at concentration range of 1 ng/ml to 1500 ng/ml. After 1 h incubation at 37° C. and washed with wash buffer for three times, goat-anti-human IgG (Fc specific)-Peroxidase was added followed by 1 h incubation at 37° C. After washed three times, TMB was added to each well and terminated by 0.5M H₂SO₄ after 5 min incubation. Absorbance at 450 nm was measured and concentration of total antibody was calculated by standard curve.

Concentration of conjugated antibody (ADC) in plasmas collected at different timepoint were determined by ELISA method: 96-well-plate was coated with Recombinant Human ErbB2 (HER2) at lug/mL for 24 h at 4° C. Then plate was blocked by 2% BSA dissolved in PBS, pH7.2 for 1 h at 37° C. After washed with wash buffer (0.05% Tween 20 in PBS pH7.2) for three times, samples with different dilution was incubated with the coated 96-well-plate, concentration of plasma was normalized to 0.1%. A standard curve prepared by ADC diluted in 0.1% plasma was also prepared at concentration range of 0.05 ng/ml to 200 ng/ml. After 1 h incubation at 37° C. and washed with wash buffer for three times, mouse-anti-vc-PAB-MMAE antibody was added followed by 1 h incubation at 37° C. After washed three times, Anti-mouse IgG (Fc specific)-Peroxidase was added followed by 1 h incubation at 37° C. Plate was wash three times and TMB was added to each well and terminated by 0.5M H₂SO₄ after 5 min incubation. Absorbance at 450 nm was measured and concentration of conjugated antibody was calculated by standard curve.

Clearance of total antibody and conjugated antibody (ADC) was shown by dashed line and solid line in FIG. 14, respectively. Plasma concentration decrease along with time for total antibodies is shown with dashed line. Plasma concentration decrease along with time for conjugated antibody (ADC) is shown with solid line. Dashed line shows similar clearance rate of total antibody for the three ADCs. Solid line indicates slower clearance of 886-16-MMAE and 886-19-MMAE compared to Trastuzumab-MMAE.

What is claimed is:

1. A polypeptide complex comprising, from N-terminus to C-terminus, a Fab domain operably linked to a hinge region, wherein the Fab domain and the hinge region or part thereof, are derived from different IgG isotypes, or part thereof.

2. The polypeptide complex of claim 1, wherein the hinge region, or part thereof, is a human IgG1, IgG2, IgG3 or IgG4 hinge region, or part thereof.

3. The polypeptide complex of claim 2, wherein the hinge region, or part thereof, is a human IgG1 or IgG4 hinge region, or part thereof.

4. The polypeptide complex of claim 1, wherein the hinge region comprises a sequence having the following formula (I):
X₁ X₂ X₃ X₄ X₅ X₆ X₇ X₈ X₉ X₁₀ CPPCP (I)
Wherein, X₁=null or E; X₂=null or P; X₃=null or K; X₄=null or S or E; X₅=null or C or S, preferably null; X₆=D or K; X₇=K or Y; X₈=T or G; and/or X₉X₁₀=HT, HP, PT or PP, Preferably PT or PP.

5. The polypeptide complex of claim 1, wherein the hinge region comprises a sequence having the following formula (II):
EPKx₁C x₂ x₃ x₄ x₅ x₆ x₇ x₈ CPPCP (II)
Wherein, x₁=null or S; x₂=null or E or S, preferably null; x₃=null or S or C; x₄=null or K or D; x₅=Y or K; x₆=G or T; and/or x₇x₈=PP, PT, HP or HT.

6. The polypeptide complex of claim 3, wherein the hinge region, or part thereof, is a human IgG1 hinge region, or part thereof.

7. The polypeptide complex of claim 6, wherein the hinge region or part thereof comprises (a) the sequence as set forth in DKTHTCPPCP (SEQ ID NO: 1) or a fragment thereof, or
(b) a sequence having at least 85% of identity to (a), or
(c) a variant of (a) or (b), wherein the variant has a mutation or mutations being selected from the group consisting of insertion, deletion and substitution, or the variant comprises a non-naturally occurring amino acid residue or non-naturally occurring amino acid residues.

8. The polypeptide complex of claim 7, wherein the hinge region, or part thereof, comprises (a) the sequence as set forth in EPKSDKTHTCPPCP (SEQ ID NO: 2) or EPKDKTHTCPPCP (SEQ ID NO: 3), or
(b) a sequence having at least 85% of identity to (a), or
(c) a variant of (a) or (b), wherein the variant has a mutation or mutations being selected from the group consisting of insertion, deletion and substitution, or the variant comprises a non-naturally occurring amino acid residue or non-naturally occurring amino acid residues.

9. The polypeptide complex of claim 6, wherein the hinge region, or part thereof, comprises (a) the sequence as set forth in any one of SEQ ID NOs: 12 to 14;
(b) a sequence having at least 85% of identity to (a), or
(c) a variant of (a) or (b), wherein the variant has a mutation or mutations being selected from the group consisting of insertion, deletion and substitution, or the variant comprises a non-naturally occurring amino acid residue or non-naturally occurring amino acid residues.

10. The polypeptide complex of claim 3, wherein the hinge region, or part thereof, is a human IgG4 hinge region, or part thereof.

11. The polypeptide complex of claim 10, wherein the hinge region, or part thereof, comprises (a) the sequence as set forth in EPKSCESKYGPPCPPCP (SEQ ID NO: 4) or a fragment thereof, or
(b) a sequence having at least 85% of identity to (a), or
(c) a variant of (a) or (b), wherein the variant has a mutation or mutations being selected from the group consisting of insertion, deletion and substitution, or the variant comprises a non-naturally occurring amino acid residue or non-naturally occurring amino acid residues.

12. The polypeptide complex of claim 11, wherein the hinge region, or part thereof, comprises (a) the sequence as set forth in EPKSCSKYGPPCPPCP (SEQ ID No. 5), or EPKSCKYGPPCPPCP (SEQ ID No. 6), or EPKSCYGPPCPPCP (SEQ ID No. 7), or EPKSCSKYGHTCPPCP (SEQ ID No. 8), or EPKSCSKYGHPCPPCP (SEQ ID No. 9), or EPKSCSKYGPTCPPCP (SEQ ID No. 10), or
(b) a sequence having at least 85% of identity to (a), or
(c) a variant of (a) or (b), wherein the variant has a mutation or mutations being selected from the group consisting of insertion, deletion and substitution, or the variant comprises a non-naturally occurring amino acid residue or non-naturally occurring amino acid residues.

13. The polypeptide complex of claim 5, wherein the hinge region, or part thereof, comprises (a) the sequence as set forth in SEQ ID NO: 11, or
(b) a sequence having at least 85% of identity to (a), or
(c) a variant of (a) or (b), wherein the variant has a mutation or mutations being selected from the group consisting of insertion, deletion and substitution, or the variant comprises a non-naturally occurring amino acid residue or non-naturally occurring amino acid residues.

14. The polypeptide complex of claim 10, wherein the hinge region, or part thereof, comprises (a) the sequence as set forth in any one of SEQ ID Nos: 15-17, or
(b) a sequence having at least 85% of identity to (a), or
(c) a variant of (a) or (b), wherein the variant has a mutation or mutations being selected from the group consisting of insertion, deletion and substitution, or the variant comprises a non-naturally occurring amino acid residue or non-naturally occurring amino acid residues.

15. The polypeptide complex of claim 1, wherein the polypeptide complex further comprises a Fc polypeptide which is operably linked to the hinge region, or wherein the polypeptide complex further comprises an additional polypeptide which is operably linked to the hinge region.

16. The polypeptide complex of claim 15, wherein the Fc polypeptide is a human IgG1, IgG2, IgG3 or IgG4 Fc polypeptide.

17. The polypeptide complex of claim 15 or 16, wherein the Fc polypeptide is a human IgG1 or IgG4 Fc polypeptide.

18. An antibody drug conjugate comprising a polypeptide complex according to claims 1-17.

19. A pharmaceutical composition comprising an antibody drug conjugate according to claim 18 and a pharmaceutically acceptable carrier or excipient.

20. A kit comprising a polypeptide complex according to claims 1-17 or an antibody drug conjugate according to claim 18 or a pharmaceutical composition according to claim 19.

21. A method of preparing the antibody drug conjugate of claim 18, comprising:
providing the polypeptide complex of any one of claims 1-17;
reacting a maleimido or haloacetyl moiety with free thiol group in cysteine residue generated by reduction of interchain disulfide bonds via Michael addition reaction.

22. The method of claim 21, wherein the free thiol group is generated by partial reduction of interchain disulfide bonds with mild reducing reagent such as TCEP or DTT, preferably the partial reduction is carried out in a buffer with pH range from 4.0 to 8.0, with reducing agent/mAb ratio from 3 to 10, reaction temperature from 4° C. to 37° C., and reduction time from 1 hr to 24 hr.

23. The method of claim 22, wherein the partial reduction is carried out with TCEP/mAb ratio from 3 to 10.

24. The method of claim 22, wherein the conjugation is carried out in a buffer with pH range from 4.0 to 8.0, organic additive from 0.0% to 20.0% by weight, drug/mAb ratio from 7 to 20, reaction temperature from 4° C. to 37° C., and conjugation time from 1 hr to 4 hr.

25. Use of the polypeptide complex of any of claims 1-17 for manufacturing an antibody drug conjugate.

26. A method of treating a condition in a subject in need thereof, comprising administrating to the subject a therapeutically effective amount of the antibody drug conjugate of claim 18.


SEQUENCE LISTING (31 sequences)

SEQ ID NO: 1 - protein, 10 aa, Artificial Sequence
  Modified hinge region
DKTHTCPPCP

SEQ ID NO: 2 - protein, 14 aa, Artificial Sequence
  Modified hinge region
EPKSDKTHTCPPCP

SEQ ID NO: 3 - protein, 13 aa, Artificial Sequence
  Modified hinge region
EPKDKTHTCPPCP

SEQ ID NO: 4 - protein, 17 aa, Artificial Sequence
  Modified hinge region
EPKSCESKYGPPCPPCP

SEQ ID NO: 5 - protein, 16 aa, Artificial Sequence
  Modified hinge region
EPKSCSKYGPPCPPCP

SEQ ID NO: 6 - protein, 15 aa, Artificial Sequence
  Modified hinge region
EPKSCKYGPPCPPCP

SEQ ID NO: 7 - protein, 14 aa, Artificial Sequence
  Modified hinge region
EPKSCYGPPCPPCP

SEQ ID NO: 8 - protein, 16 aa, Artificial Sequence
  Modified hinge region
EPKSCSKYGHTCPPCP

SEQ ID NO: 9 - protein, 16 aa, Artificial Sequence
  Modified hinge region
EPKSCSKYGHPCPPCP

SEQ ID NO: 10 - protein, 16 aa, Artificial Sequence
  Modified hinge region
EPKSCSKYGPTCPPCP

SEQ ID NO: 11 - protein, 16 aa, Artificial Sequence
  Modified hinge region
EPKCESKYGPPCPPCP

SEQ ID NO: 12 - protein, 15 aa, Artificial Sequence
  Modified hinge region
EPKSCDKTPPCPPCP

SEQ ID NO: 13 - protein, 15 aa, Artificial Sequence
  Modified hinge region
EPKSCDKTHPCPPCP

SEQ ID NO: 14 - protein, 15 aa, Artificial Sequence
  Modified hinge region
EPKSCDKTPTCPPCP

SEQ ID NO: 15 - protein, 12 aa, Artificial Sequence
  Modified hinge region
ESKYGHTCPPCP

SEQ ID NO: 16 - protein, 12 aa, Artificial Sequence
  Modified hinge region
ESKYGHPCPPCP

SEQ ID NO: 17 - protein, 12 aa, Artificial Sequence
  Modified hinge region
ESKYGPTCPPCP

SEQ ID NO: 18 - protein, 15 aa, Homo sapiens
  Hinge region of human IgG1
EPKSCDKTHTCPPCP

SEQ ID NO: 19 - protein, 12 aa, Homo sapiens
  Hinge region of human IgG4 with S228P mutation
ESKYGPPCPPCP

SEQ ID NO: 20 - protein, 214 aa, Artificial Sequence
  Amino acid sequence of the light chain of antibody 886-16
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 451 aa, Artificial Sequence
  Amino acid sequence of the heavy chain of antibody 886-16
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCSKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 213 aa, Artificial Sequence
  Amino acid sequence of the light chain of antibody 886-17
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 452 aa, Artificial Sequence
  Amino acid sequence of the heavy chain of antibody 886-17
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCSKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 214 aa, Artificial Sequence
  Amino acid sequence of the light chain of antibody 886-18
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 450 aa, Artificial Sequence
  Amino acid sequence of the heavy chain of antibody 886-18
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCSKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 214 aa, Artificial Sequence; identical to SEQ ID NO: 20
  Amino acid sequence of the light chain of antibody 886-19

SEQ ID NO: 27 - protein, 451 aa, Artificial Sequence
  Amino acid sequence of the heavy chain of antibody 886-19
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCSKYGPPCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 213 aa, Artificial Sequence; identical to SEQ ID NO: 22
  Amino acid sequence of the light chain of antibody 886-20

SEQ ID NO: 29 - protein, 452 aa, Artificial Sequence
  Amino acid sequence of the heavy chain of antibody 886-20
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCSKYGPPCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 214 aa, Artificial Sequence; identical to SEQ ID NO: 24
  Amino acid sequence of the light chain of antibody 886-21

SEQ ID NO: 31 - protein, 450 aa, Artificial Sequence
  Amino acid sequence of the heavy chain of antibody 886-21
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCSKYGPPCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK